(19) United States
(12) Patent Application Publication
(10) Pub. No.: US 2010/0009352 A1
(43) Pub. Date: Jan. 14, 2010

(54) Method for Modeling a Disease

(76) Inventors: Albert H. Gough, Glenshaw, PA (US); Patricia Johnston, Sewickly, PA (US); Kenneth A. Giuliano, Pittsburgh, PA (US); D. Lansing Taylor, Pittsburgh, PA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/227,388

(22) PCT Filed: May 24, 2007
(86) PCT No.: PCT/US2007/012406

§ 371 Date: Jul. 10, 2009

Related U.S. Application Data
(60) Provisional application No. 60/808,086, filed on May 24, 2006.

(51) Int. Cl. C12Q 1/68; C12Q 1/02

(52) U.S. Cl. 435/6; 435/29

(57) ABSTRACT

The invention described herein provides for methods of profiling cellular models of disease. Cellular systems biology is the investigation of the integrated and interacting networks of genes, proteins, and metabolites that are responsible for normal and abnormal cell functions. Methods and reagents for the profiling a disease state, the treatment of a disease state and assaying of treatments of a disease state are provided.

### RELATED APPLICATION

This application claims the benefit of U.S. Provisional Application No. 60/808,086, filed on May 24, 2006.

The entire teachings of the above applications are incorporated herein by reference.

### BACKGROUND OF THE INVENTION

Until recently, the focus in drug discovery and basic biomedical research has been on simplifying the complexity of the living human organism to individual genes, single metabolic pathways, single proteins, and one potential modulating molecule such as a small chemical compound or bioproducts to regulate complex functions. This one gene, one protein, one external modulating treatment concept dominated the drug discovery process and much of basic biomedical research for the last 15 years. This paradigm grew out of the promise of the human genome project and the theory that identifying all protein coding genes would lead to much more rapid discovery of cures for human disease (Collins et al., 2003a; Collins et al., 2003b; Phillips and Van Bebber, 2005).

Unfortunately, this reductionist approach to drug discovery has not delivered the promised efficiencies. Multiple genes, including both protein coding and non-coding genes, regulate most cellular processes (Costa, 2005; Cummins et al., 2006; Huttenhofer et al., 2005; Volinia et al., 2006), and proteins are part of complex, interacting pathways with extensive compensatory capacities. Therefore, even when a single small molecule or bioproduct has a specificity for binding to a single protein, the impact on cellular and therefore, tissue and organ function is much more complex than expected (Melnick et al., 2006). In addition, absolute specificity of small molecules and biologics are rarely demonstrated and “off target” effects must be understood for both efficacy and potential toxicity (Hopkins and Groom, 2002; Yang et al., 2004). Finally; most diseases are multi-factorial where the disease phenotype arises from the dysregulation of multiple genes, pathways and proteins (Glocker et al., 2006; Jain et al., 2005; Nadeau et al., 2003; Tuomisto et al., 2005).

Thus, a need exists to provide methods for producing, analyzing and profiling the multi-factorial processes of disease in order to more fully understand the systemic and complex interaction of disease-based cellular biology systems and to elucidate appropriate treatments.

### SUMMARY OF THE INVENTION

The cell is the simplest living system. Tissues are collections of specific cell types forming interacting colonies of cells. Although cells and tissues are less complex than a complete organism, they posses significant functional complexity allowing a detailed understanding of many aspects affecting a whole organism, such as the cellular basis of disease, treatment efficacy and potential toxicity of treatments.

The invention described herein comprises multicolor fluorescence of five or more (e.g., multiplexed) biomarkers coupled with searchable databases to provide methods for cellular systems biology profiling and analysis. Thus, the invention provides methods for analyzing and cellular systems biology (CSB) (also sometimes referred to as “systems cell biology”) profiling of cellular models of disease. Cellular systems biology is the investigation of the integrated and interacting networks of genes, proteins, and metabolites that are responsible for normal and abnormal cell functions.

In one embodiment of the invention, provided is a method for profiling a disease state. The method comprises obtaining one or more cells associated with a disease state. The one or more cells associated with a disease state are labeled with a panel of fluorescently labeled reagents, thereby producing one or more fluorescently labeled cells. Each fluorescently labeled reagent is specific for a biomarker, and the panel of fluorescently labeled reagents detects at least about five or more different biomarkers. The detection of a biomarker provides a read-out of one or more features of the one or more cells. The one or more fluorescently labeled cells are imaged with at least one optical mode to produce a first set of data. The first set of data is analyzed to read-out the features of the at least about five or more biomarkers. The combination of the features of the five or more biomarkers generates a cellular systems biology profile of the one or more cells associated with a disease state, thereby profiling a disease state.

In one embodiment, the one or more cells associated with a disease state are one or more cells associated with a cancer, a neurological disease, a metabolic disease, or an immunity-related disease.

In another embodiment, provided is a method for assessing the effect of an agent on a disease state. The method comprises contacting the one or more cells associated with a disease state with an agent to produce one or more agent-treated cells. The one or more agent-treated cells are labeled with a panel of fluorescently labeled reagents to produce one or more fluorescently labeled agent-treated cells. Each fluorescently labeled reagent is specific for a biomarker, and the panel of fluorescently labeled reagents detects at least about five or more different biomarkers. The detection of a biomarker provides a read-out of one or more features of the one or more agent-treated cells. The one or more fluorescently labeled agent-treated cells are imaged with at least one optical mode to produce a second set of data. The second set of data is analyzed to read-out the features of the at least about five or more biomarkers, wherein the combination of the features of the at least about five or more biomarkers generates a cellular systems biology profile of the one or more agent-treated cells. The cellular systems biology profile of the one or more agent-treated cells is compared with the cellular systems biology profile of the one or more cells associated with a disease state, thereby assessing the effect of an agent on a disease state.

In one embodiment, identifying similarities, differences, or combinations thereof, between the cellular systems biology profile of the one or more agent-treated cells and the cellular systems biology profile of the one or more cells associated with a disease state, indicates the effectiveness of treating the one or more cells associated with a disease state with an agent.

In another embodiment, provided is a method for profiling a cancer. The method comprises obtaining one or more cells associated with a cancer. The one or more cells associated with a cancer are labeled with a panel of fluorescently labeled reagents to produce one or more fluorescently labeled cells. Each fluorescently labeled reagent is specific for a biomarker. The panel of fluorescently labeled reagents detects eleven or more biomarkers, and the detection of a biomarker is a read-out of one or more features of a cellular systems biology profile. The one or more features can be the same or different for each biomarker detected. The one or more fluorescently labeled cells are imaged with at least one optical mode to produce a first set of data. The first set of data is analyzed to read-out the eleven or more biomarkers, and the combination of the eleven or more biomarkers generates a cellular systems biology profile of the one or more cells associated with a disease state, thereby providing a method for profiling a cancer.

In another embodiment, provided is a method for profiling Huntington's Disease. The method comprises obtaining one or more cells expressing mutant huntington protein. The one or more cells expressing mutant huntington protein are labeled with a panel of fluorescently labeled reagents to produce-one or more fluorescently labeled cells. Each fluorescently labeled reagent is specific for a biomarker, and the panel of fluorescently labeled reagents detects at least about five or more biomarkers. The detection of a biomarker is a read-out of one or more features of a cellular systems biology profile. The one or more features can be the same or different for each biomarker detected. The one or more fluorescently labeled cells are imaged with at least one optical mode to produce a first set of data. The first set of data is analyzed to read-out the at least about five or more biomarkers, wherein the combination of the five or more biomarkers generates a cellular systems biology profile of the one or more cells expressing mutant huntington protein, thereby providing a method for profiling Huntington's Disease.

In a further embodiment, provided is a method for analyzing one or more cells for the presence of a disease state. The method comprises obtaining one or more cells to test for the presence of a disease state. The one or more cells are labeled with a panel of fluorescently labeled reagents to produce one or more fluorescently labeled cells. Each fluorescently labeled reagent is specific for a biomarker. The panel of fluorescently labeled reagents detects at least about five different biomarkers, and the detection of a biomarker provides a read-out of one or more features of the one or more cells. The one or more fluorescently labeled cells are imaged with at least one optical mode to produce a set of data. The set of data is analyzed to read-out the features of the five or more biomarkers, wherein the combination of the features of the five or more biomarkers generates a cellular systems biology profile of the one or more cells. The cellular systems biology profile of the one or more cells is compared with a control cellular systems biology profile, thereby analyzing one or more cells for the presence of a disease state.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a schematic demonstrating that cells integrate many cellular processes such as gene expression, energy metabolism, etc. to yield normal functions. Diseases are due to the dysregulation of one or more of these cellular processes. Many of these processes share pathways, signals, organelles, and proteins as well as other macromolecules and metabolites which can be investigated as part of the cellular systems biology.

FIG. 2 is a schematic illustrating how cellular systems biology offers enough complexity, while allowing high throughput and cost-effective assays. Cellular systems biology and systems biology is based on the fundamental components of living systems represented by the “omics”.

FIG. 3 is a schematic of cellular systems biology profiling which involves the selection of the relevant cell types and cellular biomarkers of activity representing the cell as a system and not just a collection of targets and independent pathways. A database of responses can be used as a predictive tool in future profiles.

FIG. 4 is a schematic of particular elements of cellular systems biology profiling of cellular models of disease, including the advanced reagents and informatics tools.

FIG. 5 is a schematic demonstrating that targeting a single protein activity for drug development, such as the tumor suppressor protein p53 in cancer (center), is challenging. Within the cell, this target protein and signaling pathway is part of an integrated and interacting network. Therefore, a cellular systems biology profiling approach provides efficacy, as well as potential off-target toxicity and compensatory activities. Thus, a cell model can be constructed where specific components of the p53 pathway can have the expression levels regulated by gene switches, short interfering RNAs, or combinations of both and specific protein-protein interactions; molecular processes, and phenotypic responses can be measured as part of the cellular systems biology profile.

FIG. 6 is a graph of the regulation of the expression level of a fluorescent-hdm2 under the control of a gene switch showing the expression levels after 12, 24 and 40 hours of stimulation with the inducer.

FIG. 7 are example images of cellular feature labeling for cellular systems biology analysis. Only four example biomarkers from the eleven biomarkers used to produce the cellular systems biology profile containing 15 total features. HDM2-GFP is shown with the expressing cells overlaid with Hoechst 33342 labeled nuclei. An antibody to α-tubulin was used to measure changes in the stability of the microtubule cytoskeleton. Antibodies against APE/Ref-1 labeled the dual function enzyme that takes part in DNA repair as well as regulating the redox state of DNA-binding proteins. Changes in the nuclear distribution of APE/Ref-1 were used to measure the extent of DNA-repair pathway activation. The 14-3-3 protein, through its association with the cytoskeleton, regulates the localization and activity of other proteins (e.g., Cdc25). The phosphorylation level of cytoplasmically localized 14-3-3 protein was used as a measure of its protein-protein interaction activity.

FIG. 8 is a cellular systems biology profile of a cellular model of cancer where a human lung cancer cell line was transduced with a fluorescently tagged protein, HDM2, a modulator of p53 that was regulated with the RheoSwitch™ gene switch. The level of expression of this tagged HDM2 was regulated by increasing the concentration of the inducer ligand (increased concentration of expression on y axis). This allowed the “manipulation” of HDM2 levels in cells. The CSB profile was defined by measuring the KS statistics of 15 distinct CSB features (from eleven biomarkers, see abbreviations of biomarkers on the top with clustering) in a fixed end-point assay using imaging microscopy. The KS statistics were color coded to show those CSB features that did not change (blue), changed moderately (black) and changed extensively (yellow-red).

FIG. 9 illustrate cell maps to visualize examples of the CSB features used to produce CSB profiles demonstrating the relationships between cellular processes within the same cells. Cell maps were prepared from the cellular systems biology profiling data as described [1, 5]. Imaging microscopy measurements of histone H3 phosphorylation and APE/Ref-1 intracellular distribution made in the same cells were plotted against each other at various HDM2 expression levels that were induced with RSL-1. The fiduciary lines, which are constant in each map, show that the cells exhibiting decreased phospho-histone H3 levels were in large part the same cells that exhibited predominately nuclear localized APE/Ref-1.

FIG. 10 are example cellular systems biology profiling images for a model of Huntington's Disease (HD). PC12-HttQ103 cells were seeded in 384-well plates +/− NGF (100 ng/ml). At two days post-seeding cells were induced with 200 nM RSL1 +/− NGF (100 ng/ml) for 48 h. Following fixation, cells were stained with Hoechst and with antibodies raised against tubulin and phospho c-Jun. Tubulin was detected with a donkey anti mouse Cy3 conjugated secondary antibody and phospho c-Jun was visualized with a donkey anti rabbit Cy5 conjugated secondary antibody. Images were captured with an HCS instrument and Hoechst-stained nuclei, mutant huntingtin-GFP expression, phospho c-Jun labeled nuclei, and tubulin-stained cells are shown. To further refine a cellular systems biology profile, up to ten other cellular biomarkers, such as those specified under the additional exemplification can be applied to the analysis.

FIG. 11 is a graph illustrating that mutant huntingtin aggregation correlates with cell loss. PC12-HttQ103 cells were seeded in as described in FIG. 10 and stimulated with 200 nM RSL1 for the indicated times. Cells were stained with Hoechst and phospho-Jun-specific antibodies as described in FIG. 10. Images were captured with an HCS instrument and the cell number and % cells with aggregates were determined with standard imaging algorithms. The cell number and % cells with aggregates as a function of the RSL1 induction time are illustrated. To further refine a cellular systems biology profile, up to ten other cellular biomarkers, such as those specified under the additional exemplification can be applied to the analysis.

FIG. 12 illustrates selective c-Jun activation in mutant huntingtin-GFP aggregate-producing cells. PC12-Htt-Q103 cells were treated with 200 nM RSL1 in accordance with FIG. 11. At 48 h post-induction, cells were fixed and stained with antibodies raised against the Serine 73 phosphorylated form of c-Jun. Primary antibodies were detected with donkey anti rabbit Cy5-conjugated secondary antibodies and stained with Hoechst. Images were captured with an HCS instrument and standard imaging algorithms were used to determine the mean nuclear total intensity for the entire cell population, the non-Htt-aggregating population (−Agg), and the mutant huntingtin aggregate producing cells (+Agg). To further refine a cellular systems biology profile, up to ten other cellular biomarkers, such as those specified under the additional exemplification can be applied to the analysis.

FIG. 13 is a graph illustrating p53 activation is associated with mutant huntingtin aggregate formation PC12-Htt-Q103 cells were treated as described in FIG. 12. Cells were stained with p53-specific antiserum and visualized with donkey anti sheep Cy3-conjugated secondary antibodies. Images were captured and analyzed as described in FIG. 12. To further refine a cellular systems biology profile, up to ten other cellular biomarkers, such as those specified under the additional exemplification can be applied to the analysis.

FIG. 14. are graphs illustrating little correlation between aggregate formation and differentiation PC12-Htt-Q103 cells were treated and stained as stated in FIG. 10. Aggregate formation in untreated and NGF-treated populations was measured with standard imaging algorithms. Mean neurite count and mean branch point distance form the cell body was also measured. To further refine a cellular systems biology profile, up to ten other cellular biomarkers, such as those specified under the additional exemplification can be applied to the analysis.

FIG. 15 is graphs illustrating aggregate formation impacts the cell cycle and nuclear area. PC12-Htt-Q103 cells were treated and stained as outlined in FIG. 10. DNA content and Nuclear area are presented. To further refine a cellular systems biology profile, up to ten other cellular biomarkers, such as those specified under the additional exemplification can be applied to the analysis.

FIG. 16 is graphs of the effects that chemical compounds had on the formation of GFP-huntingtin aggregates in PC12T cells. Compounds either had no effect on GFP-huntingtin aggregate formation at multiple concentrations, or either induced or inhibited aggregate formation at one or more concentrations. Both microtubule stabilizers and destabilizers modulated GFP-Htt aggregate formation. To further refine a cellular systems biology profile, up to ten other cellular biomarkers, such as those specified under the additional exemplification can be applied to the analysis.

FIG. 17 is a series of studies showing that compound induced modulation of cell cycle regulation (e.g., 2 n/4 n distribution ratio) was itself modulated in the presence of GFP-huntingtin aggregates. For example, cells with aggregates were less responsive to the compound nocodazole than were cells with out aggregates. To further a cellular systems biology profile, up to ten other cellular biomarkers, such as those specified under the additional exemplification can be applied to the analysis.

FIG. 18 is a series of studies demonstrating the effects of nocodazole on DNA content, microtubule stability, nuclear size, and chromatin condensation are shown in cells either with GFP-huntingtin aggregates or in cells without aggregates. Nocodazole-induced cell cycle regulation and nuclear morphological changes were partially inhibited in cells containing aggregates. Furthermore, cells with aggregates exhibited greater sensitivity to the microtubule destabilizing activity of nocodazole than cells without aggregates. To further refine a cellular systems biology profile, up to ten other cellular biomarkers, such as those specified under the additional exemplification can be applied to the analysis.

FIG. 19 shows the resulting distribution maps of the effects that hKIS protein knockdown has on the intracellular distribution of over expressed wild type and mutant p27 isoforms. The p27 distribution in U2OS cells cotransfected with HA-tagged p27 isoforms and hKIS siRNAs was dependent on the p27 expression level, but independent of the isoforms of the p27 being over expressed and also independent of the knockdown of endogenous hKIS using siRNAs. To further refine a cellular systems biology profile, up to ten other cellular biomarkers, such as those specified under the additional exemplification can be applied to the analysis.

FIG. 20 is a series of studies distribution maps of the effects that hKIS protein knockdown has on the regulation of the cell cycle, specifically the DNA content, in cells with over expressed wild type and mutant p27 isoforms. The DNA content in U2OS cells cotransfected with HA-tagged p27 isoforms and hKIS siRNAs was dependent on the p27 expression level, but independent of the isoforms of the p27 being over expressed and also independent of the knockdown of endogenous hKIS using siRNAs. To further refine a cellular systems biology profile, up to ten other cellular biomarkers, such as those specified under the additional exemplification can be applied to the analysis.

### DETAILED DESCRIPTION OF THE INVENTION

Drug discovery has come to rely on high throughput screening (HTS) technologies that focus on isolated targets as a means to boost productivity. Technologies such as high content screening (HCS) have been introduced that combine some of the efficiencies of HTS with biological context of the intact cell. HCS makes it possible to determine the effect of experimental compounds on target proteins within the context of the living cell. However, the focus of HCS continues to be on individual target protein activities. Cellomics, Inc. introduced the first HCS platform for drug discovery in 1997 and enabled the field of cellomics (see Giuliano et al., 1997; Taylor, 2006a; Taylor, 2006b). The discipline of cellomics can be described as the first step towards understanding how the output of the foundational “omics” technologies, i.e., genomics, proteomics and metabolomics function in a living environment (Giuliano et al., 1997; Giuliano et al., 2003; Taylor, 2006a). But cellomics and the target-focused use of HCS falls short of elucidating cellular activity as the function of a system.

“Systems biology” is the study of a whole organism viewed as an integrated and interacting network of genes, proteins and metabolic reactions which give rise to life (Aloy and Russell, 2005; Bugrim et al., 2004; Butcher et al., 2004; Hood and Perlmutter, 2004; Westerhoff and Palsson, 2004). Thus, the various disciplines of “omics” (e.g., genomics, proteomics, metabolomics) focus on the parts of a system, while systems biology deals with the functional complexity of the whole. The “omics” approach is an over simplification, while systems biology at the organismal level is a relatively low throughput and expensive process.

Provided herein are methods of generating a cellular systems biology profile of one or more cells. The one or more cells can be cells associated with a disease state (e.g., the disease state can be a cancer, a neurological disease, a metabolic disease, or an immunity-related disease), or isolated from a diseased tissue (e.g., a cancerous tissue, a nerve tissue including those tissues in the peripheral and central nervous systems, blood cells, including peripheral blood cells, white blood cells, and the like), or isolated from a tissue or blood of an animal known to be healthy, an animal suspected to have a disease, an animal at risk of a disease, an animal treated for a disease, or an animal at different time points (e.g., after treatment for a disease, for monitoring a disease, for monitoring treatment of a disease, etc.). The tissue (e.g., diseased tissue) can be from an animal. In one embodiment the animal is a human.

In one embodiment, provided herein are methods of generating a cellular systems biology profile of one or more cells associated with a disease state. Thus, the methods provided herein can be used to profile a disease. Such methods comprise generating a cellular systems biology profile of one or more cells associated with a disease state. Cells associated with a disease state include one or more cells that are isolated from a diseased tissue and/or a diseased animal, such as a human. In addition, cells associated with a disease state can be cells (e.g., wild type or normal cells) that have been manipulated to exhibit one or more disease phenotypes. Generally, the methods comprise obtaining one or more cells associated with a disease state. The one or more cells associated with a disease state are labeled with a panel of fluorescently labeled reagent to produce fluorescently labeled cells. Each fluorescently labeled reagent is specific for a biomarker. In one embodiment, the panel of fluorescently labeled reagents detects at least about five different biomarkers. The detection of a biomarker provides a read-out of one or more features of the one or more cells. The method can further comprise imaging the fluorescently labeled cells with at least one optical mode to produce a set of data. The set of data is analyzed to read-out the features of the at least five biomarkers, such that the combination of five or more biomarkers generates a cellular systems profile of the one or more cells associated with a disease state, thereby profiling the disease state.

As used herein, a “biomarker” is a cellular constituent and/or activity (e.g., protein or other macromolecules, organelles, ions, metabolites, etc.) that can be specifically “labeled” with a fluorescence based reagent (also referred to herein as a fluorescently labeled reagent) (including, but without limitation, fluorescently labeled antibodies, fluorescently labeled peptides, fluorescently labeled polypeptides, fluorescent protein biosensors, fluorescently labeled aptamers, fluorescently labeled nucleic acid probes, fluorescently labeled chemicals, and fluorescent chemicals). A variety of fluorescently labeled reagents and methods for use of such fluorescently labeled reagents to detect a biomarker are known to those of skill in the art. Fluorescently labeled reagents can be obtained from commercial sources (e.g., Molecular Probes). In one embodiment, the panel of fluorescently labeled reagents detects at least about five different biomarkers.

The detection of a biomarker in one or more cells is a read-out of one or more features of the cells or tissue. As used herein, a “feature” is a measurement (e.g., image-based measurement) or series of measurements of a particular biomarker that include, but not limited to measurements of morphometry, intensity, fluorescence anisotropy, fluorescence lifetime, localization, ratios and/or differences, that can indicate a biological function or activity of the cell. Biological functions indicated by biomarkers include, but are not limited to: protein posttranslational modifications such as phosphorylation, proteolytic cleavage, methylation, myristoylation, and attachment of carbohydrates; translocations of ions, metabolites, and macromolecules between compartments within or between cells; changes in the structure and activity of organelles; and alterations in the expression levels of macromolecules such as coding and non-coding RNAs and proteins, morphology, state of differentiation, and the like. A single biomarker can provide a read-out of more than one feature. For example, Hoechst dye can be used to detect DNA (e.g., a biomarker), and a number of features of the cells (e.g., nucleus size, cell cycle stage, number of nuclei, presence of apoptotic nuclei, etc.) can be identified by the DNA detected with the Hoechst dye.

“Cellular systems biology” (also referred to herein as “CSB” or “Systems Cell Biology”), is the investigation of the integrated and interacting networks of genes, proteins, and metabolites that are responsible for normal and abnormal cell functions. Thus, a “cellular systems biology profile” is a systemic characterization of the interactions, relationships, and/or state of the constituents of cells as indicated by at least about five or more biomarkers that give rise to the cellular systems biology features that are used to construct the profile. The interrelationships within a cellular systems biology profile are defined, for example, either arithmetically (e.g., ratios, sums, or differences between cellular systems biology feature values) or statistically (e.g., hierarchical clustering methods or principal component analyses of combinations of cellular systems biology feature values).

Cellular systems biology harnesses the higher throughput capacity of automated microscopy technologies while avoiding the expense and potential confounding species related problems (worms, flies, fish) associated with traditional organism-based systems biology (Giuliano et al., 2006b). A cellular systems biology profile is a systemic characterization of cells in the context defined as the study of the living cell, the basic “unit of life”; an integrated and interacting network of genes, proteins and a myriad of metabolic reactions that give rise to function (Giuliano et al., 2005; Taylor, 2006a; Taylor and Giuliano, 2005).

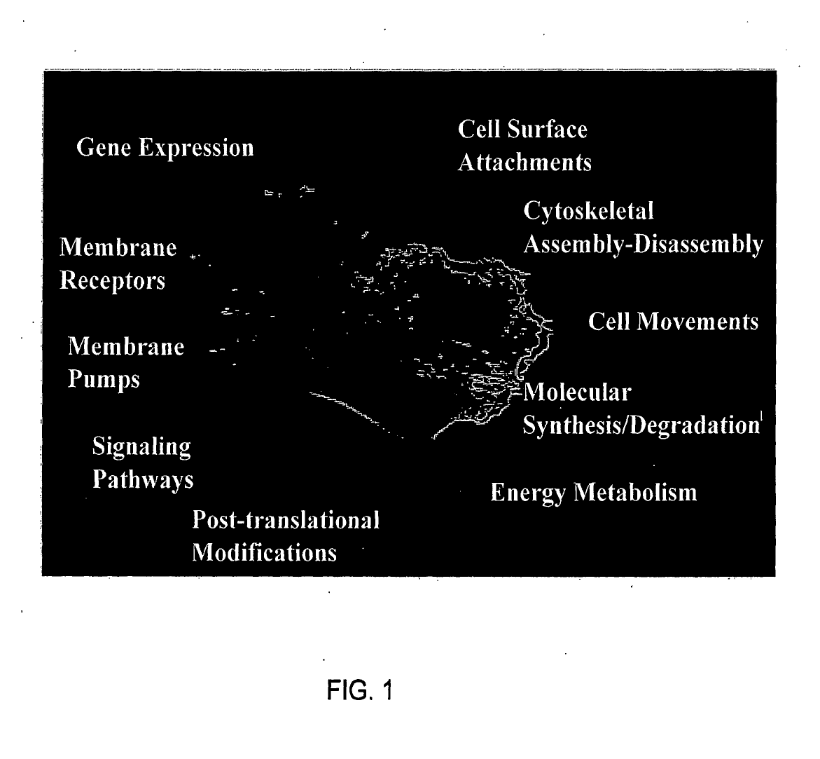

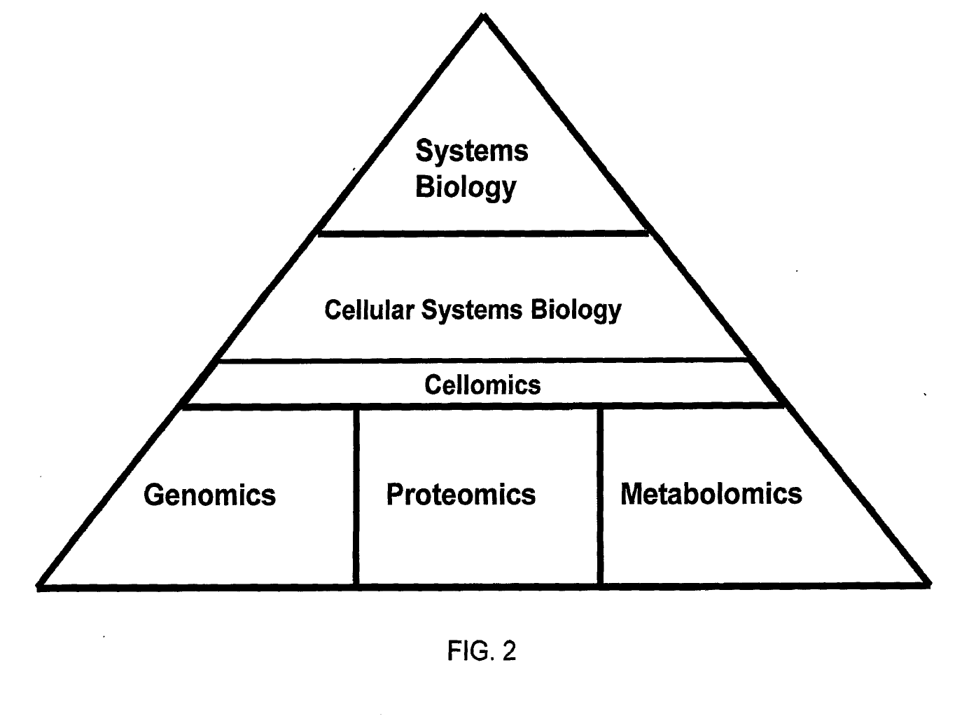

One can think of the cell as the simplest living “system” (FIG. 1): much less complex than a complete organism, but possessing the systemic functional complexity that facilitates a detailed understanding of the response of the integrated system to a perturbant or agent such as a drug. The cell and combinations of cells therefore serve as models in which to explore drug efficacy and potential toxicity, inexpensively and rapidly, across a broad range of doses and response times. Selection of the optimal cell types, whether primary cells or cell lines (e.g., vertebrate or invertebrate cells or cell lines, mammalian cells or cell lines, human cells or cell lines, and the like), coupled with focused cellular systems biology profiling assays, reagents; and informatics analysis is fundamental to making cellular systems biology a valuable approach to drug discovery and development, as well as biomedical research and diagnostics (FIG. 2).

Cellular systems biology models elucidate the impact of small molecules and bioproducts on cell functions by characterizing the cellular responses with a large number (e.g., at least about five to about fifteen, from at least about five to about twelve, from at least about five to about ten, from at least about five to about eight biomarkers (see, e.g., Giuliano et al., 2005; Taylor and Giuliano, 2005). Multiplexing facilitates simultaneous capture of many biomarkers of cellular function and informatics tools enable identification of the interaction of all the biomarkers. These cellular responses and complex interactions can be related to a set of cellular function biomarkers: a unique cellular systemic profile.

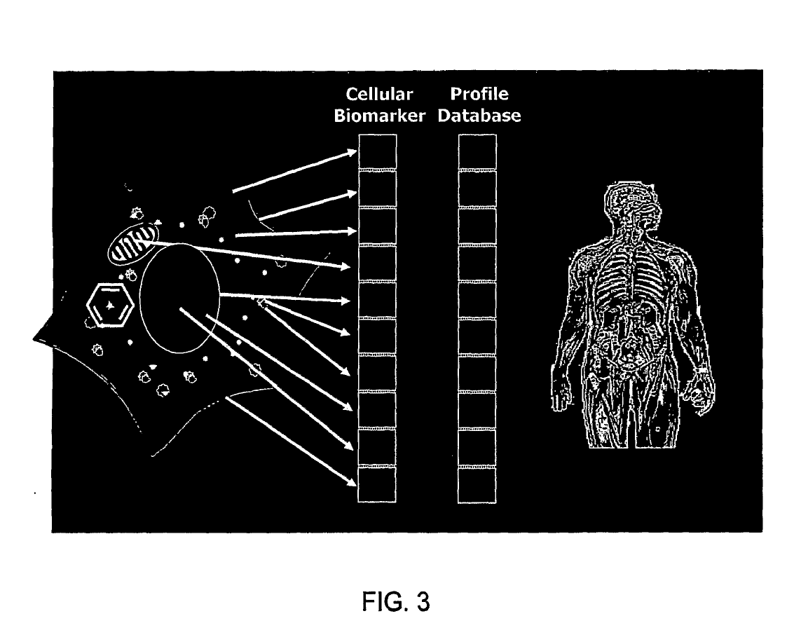

The cellular function biomarkers selected to represent the systemic response of particular cells are measured with a variety of fluorescence-based reagents (e.g., a fluorescence-based reagent that detects one or more biomarkers). The response profile of a variety of human cells to perturbants or agents can be used to build a database that can be mined to determine the effect of “new” perturbants or agents to those of known cellular mechanism (see, e.g., FIG. 3).

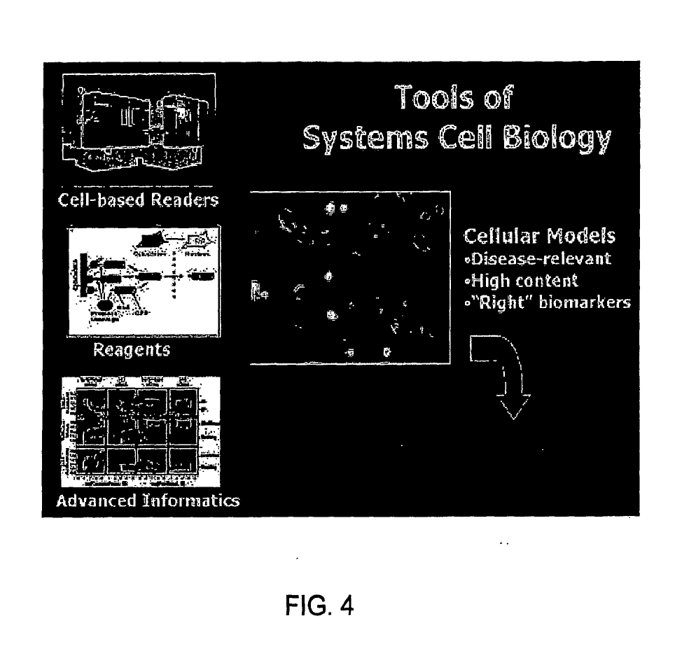

As shown herein, cellular systems biology profiling can use imaging microscopy platforms to yield data on the response of individual cells, as well as their sub-cellular responses. Cellular systems biology leverages the power of imaging microscopy technologies, which comprise readers, first-generation reagents and basic informatics software, with panels of cellular function reagents, including more reagents that can dynamically measure and manipulate cellular constituents. In addition, cellular systems biology profiling informatics are used to generate new systems knowledge of disease, as well as profiles of the cellular systemic response to emerging drugs (see, e.g., FIG. 4).

There are now several commercially available HCS instruments and application software packages, as well as sophisticated imaging microscopy systems compatible with cellular systems biology (Gough and Johnston, 2006).

Imaging microscopy methods and HCS assays, begin with living cells that are treated with small molecules, bioproducts and/or physical disruption. Cells are fixed at several time points and subsequently labeled and read on an imaging reader.

These assays reflect cellular activity at a moment in time and are known as “fixed endpoint assays”. Sample preparation methods are available to automate all of these steps making the assays fast and reproducible. Thus, the fixed endpoint approach can be a relatively high throughput screening method, even for cellular systems biology. However, the time domain of the biology is limited to one time point. Therefore, the investigator must either create a time series by preparing multiple plates processed over time or initially define the half-time of some cellular process of interest and set the time of fixation accordingly.

In one embodiment provided herein, the one or more cells are live cells. Live cell imaging microscopy is possible with the integration of on-board fluidics environmental chamber (Abraham et al., 2004b; O'Brien and Haskins, 2006; O'Brien et al., 2006). This can be accomplished with an integrated platform (see Gough and Johnston, 2006, for a review of HCS screening platforms) or by applying add-ons to a fixed endpoint reader and/or other imaging microscopy system. Live cell imaging microscopy assays or cellular systems biology profiles can be based on a single time point measurement using fluorescence-based reagents that are functional in living cells, or the assays can be comprised of multiple kinetic measurements that are made starting before the experimental treatment and continuing over time. Kinetic assays are useful in defining the half-times of specific biological processes and for critically defining the complex temporal-spatial dynamics of cells and their processes.

In another embodiment provided herein is a method for profiling a disease state, which comprises obtaining one or more cells associated with a disease state. As described herein, the one or more cells associated with a disease state (e.g., the disease state can be a cancer, a neurological disease, a metabolic disease, or an immunity-related disease) can be isolated from a diseased tissue (e.g., a cancerous tissue, a nerve tissue including those tissues in the peripheral and central nervous systems, blood cells, including peripheral blood cells, white blood cells, and the like). The diseased tissue can be from an animal. In one embodiment the animal is a human. Methods for isolating one or more cells from a diseased tissue or from a diseased animal are well known in the art, and include, for example, biopsy, excision, venipuncture, dissociation of tissue samples, etc.

Alternatively, the one or more cells can be cells manipulated to exhibit one or more disease phenotypes (e.g., such as those phenotypes exhibited by cells associated with a cancer, a neurological disease, a metabolic disease, or an immunity-related disease). Such cells can be cell lines, primary cells, and the like. Cells can be manipulated to exhibit one or more disease phenotypes using any methods standard in the art, including gene transfection, protein expression, siRNA knockdown, receptor-mediated endocytosis, chemical modification, and the like. In one embodiment, one or more molecules can be introduced into a cell (e.g., intracellularly, onto the surface of a cell) using standard methods. Such methods can be used to produce one or more cells exhibiting a disease phenotype. The one or more molecules can be selected from the group consisting of: DNA, RNA, protein, aptamer, peptide tag, carbohydrate, lipid, and a combination thereof. As will be understood by a person of skill in the art, the DNA can be single stranded, double standed, a cDNA, etc. An aptamer is oligonucleic acid or peptide molecules that bind a specific target molecule. Peptide tags are peptide molecules encoding fragments of modulatory proteins or epitopes for further labeling and can encode membrane transport peptides that enable translocation across a membrane. In one embodiment, the one or more molecules are introduced into one or more cells in order to manipulate the cells to express a disease phenotype. In one embodiment, the one or More molecules are constitutively or conditionally expressed. In a further embodiment, the one or more molecules are conditionally expressed in the one or more cells. As will be understood by a person of skill in the art, conditional expression of one or more molecules in a cell can be achieved by any suitable method, such as drug responsive expression (e.g., gene expression controlled by tetracycline, ecdysone, and cumate, or their derivatives).

In one embodiment, the one or more cells are associated with a cancer. In another embodiment, the one or more cells are associated with cancer and are manipulated to express an increased level of p53 as compared to normal cells. In a further embodiment, the one or more cells are associated with a neurological disease. In one embodiment, the one or more cells are associated with Huntington's Disease. In another embodiment, the one or more cells are associated with Huntington's Disease and are manipulated to express a mutated Huntingtin protein (e.g., Htt).

The method further comprises labeling the one or more cells associated with a disease state with a panel of fluorescently labeled reagents to produce one or more fluorescently labeled cells. In one embodiment, the panel of fluorescently labeled reagents is selected from the group consisting of fluorescently labeled antibodies, fluorescently labeled peptides, fluorescently labeled polypeptides, fluorescent protein biosensors, fluorescently labeled aptamers, fluorescently labeled nucleic acid probes, fluorescently labeled chemicals, fluorescent chemicals, and combinations thereof. In one embodiment, the fluorescently labeled reagent is a fluorescently labeled RNA. Each fluorescently labeled reagent is specific for at least one biomarker, and the panel of fluorescently labeled reagents detects at least about four, five, six, seven, eight, nine, ten, or more different biomarkers. Each fluorescently labeled reagent can be identified by its particular fluorescence characteristics (e.g., wavelength for excitation and/or emission, intensity, fluorescence anisotropy, fluorescence lifetime, and the like). The detection of a biomarker provides a read-out of one or more features the cells. For example, the panel of fluorescently labeled reagents can indicate the presence, amount, location, activity, distribution, or combination thereof, of the biomarkers in the one or more fluorescently labeled cells.

In one embodiment, the method further comprises imaging the one or more fluorescently labeled cells with at least a one optical mode to produce a first set of data. Imaging can be performed using any suitable means, for example, wide field microscopy or confocal microscopy. In one embodiment, at least one optical mode is fluorescence light microscopy. Data that are produced from imaging can be visual or digital. In one embodiment, the set of data are digital data.

In one embodiment, the method further comprises analyzing the data to read-out the features of the at least about five or more biomarkers, wherein the combination of the at least about five or more biomarkers generates a cellular systems biology profile of the one or more cells associated with a disease state, thereby providing a method for modeling a disease state. In one embodiment, the combination of at least about four, five, six, seven, eight, nine, ten or more features generates a cellular systems biology profile.

In a further embodiment, the cellular systems biology profile is stored in a database for reference, thereby providing a reference cellular systems biology profile in a database. In one embodiment, the database is a computer. In another embodiment, the database is stored on a server. In one embodiment, the reference cellular systems biology profile in the database is compared with a cellular systems biology profile of a test sample of one or more cells. A test sample can be a sample of healthy cells, wild-type cells, cells from an animal (e.g. a human) at risk of a disease, a sample from an animal (e.g., a human) after treatment with an agent or treatment of a disease (e.g., to monitor disease progression, disease recovery, disease relapse, and the like). In one embodiment, the cellular systems biology profile of a normal cell is a reference cellular systems biology profile. A normal cell includes a wild-type cell, or a healthy cell, or a diseased cell that has been successfully treated to eliminate or alleviate the disease. The comparison of cellular systems biology profiles permits the identification of similarities, differences, or a combination thereof, between the cellular systems biology profiles being compared. Various methods can be used to compare two or more cellular systems biology profiles, such as by graphical display, cluster analysis, or statistical measure of correlation and combinations thereof. In one embodiment, the method comprises comparing the cellular systems biology profiles of the one or more cells associated with a disease state a before and after the conditional expression of the one or more molecules in the one or more cells. In another embodiment, the method comprises comparing the cellular systems biology profiles of the one or more cells associated with a disease state before and after treatment with an agent. Thus, in one embodiment, the comparison of cellular systems biology profiles of the one or more cells associated with a disease state before and after treatment with an agent monitors the treatment with an agent. In a further embodiment, the method comprises comparing the cellular systems biology profiles of the one or more cells associated with a disease state at different time points, wherein one or more cells associated with a disease state are obtained at one or more time points. In another embodiment, the method comprises comparing the cellular systems biology profiles of the one or more cells associated with more than one disease states.

Thus, in one embodiment, the method further comprises obtaining two or more cells associated with two or more disease states, profiling the cellular systems biology of the two or more disease states and comparing the cellular systems biology profiles of two or more cells associated with the two or more disease states. In another embodiment, two or more samples of cells associated with a disease state are obtained from an animal at two or more time points, wherein at least sample of one or more cells are obtained from each time point. The method comprises profiling the disease state of the one or more cells obtained from each time point, thereby generating a cellular systems biology profile of the one or more cells obtained from each time point and comparing the cellular systems biology profiles of the one or more cells obtained from each time point. Thus, the cellular systems biology profiles of the one or more cells can be compared at a first, second, third, or more time points.

In another embodiment, provided herein is a method for assessing the effect of an agent on a disease state. In one embodiment, the agent is a drug. The one or more cells associated with a disease state are contacted with an agent to produce one or more agent-treated cells. The one or more agent-treated cells are labeled with a panel of fluorescently labeled reagents to produce one or more fluorescently labeled agent-treated cells. Each fluorescently labeled reagent is specific for a biomarker. The panel of fluorescently labeled reagents detects at least about five different biomarkers. The detection of a biomarker provides a read-out of one or more features of the-one or more agent-treated cells. The one or more fluorescently labeled agent-treated cells are imaged with at least one optical mode to produce a second set of data. The second set of data is analyzed to read-out the features of the five or more biomarkers, and the combination of the features of the at least about five or more biomarkers generates a cellular systems biology profile of the one or more agent-treated cells. The cellular systems biology profile of the one or more agent-treated cells is compared with the cellular systems biology profile of the one or more cells associated with a disease state, thereby assessing the effect of an agent on a disease state. Identifying similarities, differences, or combinations thereof, indicates the effectiveness of treating the one or more cells associated with a disease state with an agent. In another embodiment, the cellular systems biology profile of the one or more agent-treated cells is compared with a suitable control (e.g., a control cellular systems biology profile). In any of the methods described herein, a cellular systems biology profile pan be compared with a control cellular systems biology profile. A control can be the cellular systems biology profile of a wild-type cell, a normal cell, a known diseased cell, a reference cell, a cell before contact with an agent, a cell without contact with an agent, a cell with varying amounts of agent contacted with the cell, etc.

In a further embodiment, provided are methods for diagnosis. In one embodiment, the method analyzes one or more cells for the presence of a disease state. In a particular embodiment, the method comprises obtaining one or more cells to test for the presence of a disease state. The one or more cells are labeled with a panel of fluorescently labeled reagents to produce one or more fluorescently labeled cells. Each fluorescently labeled reagent is specific for a biomarker. The panel of fluorescently labeled reagents detects at least about five different biomarkers, and the detection of a biomarker provides a read-out of one or more features of the one or more cells. The one or more fluorescently labeled cells are imaged with at least one optical mode to produce a set of data. The set of data is analyzed to read-out the features of the five or more biomarkers, wherein the combination of the features of the five or more biomarkers generates a cellular systems biology profile of the one or more cells. The cellular systems biology profile of the one or more cells is compared with a control cellular systems biology profile, thereby analyzing one or more cells for the presence of a disease state. In one embodiment, the control cellular systems biology profile is the cellular systems biology profile of one or more cells associated with a disease state. In another embodiment, the control cellular systems biology profile of one or more cells associated with a disease state is stored in a database. In one embodiment, this method is used to monitor the treatment of an animal (e.g., an animal with a disease state, an animal at risk for a disease state, and the like).

In one embodiment, one or more steps of the methods are automated. For example, the imaging procedures are automated. Furthermore, analyzing the data can be performed manually, by automation or a combination thereof. In some embodiments, the method is wholly automated.

In a further embodiment, reagents for the selective and dynamic measurement and manipulation of cellular constituents and responses are listed in Table I (below).

TABLE I
Examples of reagents used to measure and manipulate cellular
constituents (see, e.g., Giuliano et al., 2006a).

| Selected Reagents to Measure | Selected Reagents to Manipulate |
|---|---|
| Biomarkers | Cellular Activities |
| Antibodies | Directed siRNA to coding RNA |
| Fluorescent protein tagging | Random siRNA |
| Diffusible fluorescent probes | Gene switches |
| Fluorescent protein biosensors | Caged compounds |

These reagents can be used in the cellular models of disease analyzed by cellular systems biology profiling.

Multiple reagents exist for the measurement of multiple biomarkers based on fluorescence and more biomarkers can be analyzed by using, for example, more than one cell at more than one location on a plate (e.g., as will be appreciated by a person of skill in the art, the plate can be any suitable plate for cells, such as a microplate, e.g., with multiple wells, a slide, or the more than one cells can be on an array such as a microarray, etc.) to profile the same treatment. In one embodiment, when using more than one cell at more than one location to profile a particular treatment, redundant measurements of some of the same biomarkers in each of the one or more locations of one or more cells can be performed. In one embodiment, the redundant measurements of one or more biomarkers is used to confirm the uniform treatments of one or more cells in the one or more location. In addition, this method can also be used to build relationships (such as of profiles) between biomarker measurements even though they are not measured in the same cells. In one embodiment, two or more cells associated with a disease state are located at two or more positions on a plate. The read-out of the features of five or more biomarkers is generated from the two or more cells located at the two or more positions on the plate, and at least one biomarker is the same biomarker in the two or more cells located at two or more positions on the plate. In a further embodiment, the one or more cells associated with a disease state are presented in an array. For example, the array can be a multi-well array, such as a 96 well, a 396 well, or a 1084 well format. The read-out of the features of the five or more biomarkers can be generated from one or more cells in one or more positions (or wells) in the array. In a particular embodiment, one or more biomarkers of the one or more cells in each well are the same, thereby providing a direct measure of the same cellular function Or state of the one or more cells in each of the positions (or wells) in the array.

There are many choices of reagents to measure cellular responses. For example, antibodies are readily available for a large number of targets and can be multiplexed with multicolored fluorescent dyes for imaging microscopy assays on the same populations of cells. Fluorescent proteins such as the green fluorescent protein from the jelly fish, as well as alternative versions, can be used to express fluorescently tagged target proteins in cells. Diffusible fluorescent probes are available that can label specific organelles and monitor a variety of cellular metabolites and ions. Some of the diffusible probes can be fixed for end point measurements. Fluorescent protein biosensors can be created to detect numerous protein activities through changes in the fluorescence properties (e.g., intensity, spectral shifts, anisotropy, lifetime, etc.). As used herein, a “fluorescent protein biosensor” is a fluorescently labeled protein that is used to measure the activities of macromolecules, metabolites, and ions within cells.

In a further embodiment, a number of reagents can be used to manipulate cell constituents in such a manner as to produce a disease phenotype. For example, directed siRNAs have can be used to “knock down” the expression of specific proteins in particular pathways and disease states. Non-coding RNA's such as micro-RNA's can also be used to Manipulate cellular processes. Gene switches can be used to regulate the expression of either proteins or siRNA's. Gene switches permit the on/off, up-regulated/down-regulated control of gene expression. Multiple gene switches in the same cells permit more sophisticated manipulations of cellular processes. Caged compounds are molecules whose normal activity is under control, for example, of illumination at a specific wavelength of light to release the activity within cells in time and space.

In still a further embodiment, cellular systems biology profiles are produced from panels of molecularly specific biomarkers with broad coverage of cell functions. The methods for producing these profiles comprises: multiplexing of biomarker measurements; correlating features derived from biomarkers within single cells; analysis of subpopulations of cells; clustering and pattern matching of multifactorial data sets; and modeling cellular systems. Furthermore, methods are disclosed to correlate a diversity of features in single cells. Rather than reducing the cell population response to a single value such as the mean or median of the measurement, other methods of cell population analysis can be used. For example, cell cycle analysis can be performed by analyzing the population and segmenting it into subpopulations, which may respond differentially. Direct comparison of population distributions, for example by making use of the Kolomogorov-Smirnov test, provides a much more sensitive measure of the shift in a population response than the use of standard statistical parameters like the mean or median of the distribution.

In another embodiment, Network modeling methods can be used in extracting more information from cellular systems biology profiles (see, e.g., Araujo and Liotta, 2006 and Janes and Lauffenburger, 2006). These models can, for example, be used as a tool for diagnostics and used as surrogate endpoints for therapies (Danna and Nolan, 2006).

In another embodiment, cellular systems biology profiles represent the cellular responses across many assays for a specific disease model or to a Compound treatment. These profiles can be mined to identify correlations between response profiles, which provide a means to predict functional effects, including detailed disease mechanism or mode of action of a particular treatment.

### EXAMPLE 1

### Cellular Systems Biology Profiling Model of Cancer Model Using the Human Lung Carcinoma Cell Line (A549) Expressing Wild Type p53

In anticancer drug discovery, the myriad cellular events regulated by the p53 tumor suppressor protein present an invaluable set of potential for imaging microscopy targets [1]. That the mutation or deletion of p53 protein in many cancer cell types is often a determining factor in the chemotherapeutic outcome [2-4], emphasizes the need for information on the cellular and molecular activities. regulated by p53 protein and the effect drugs have on these interrelationships. Thus, there is a need for new approaches to rapidly and precisely modulate components of the p53 signaling pathway, as well as complementary cellular systems biology methods to dissect the network of cellular and molecular activities regulated by this important tumor suppressor protein.

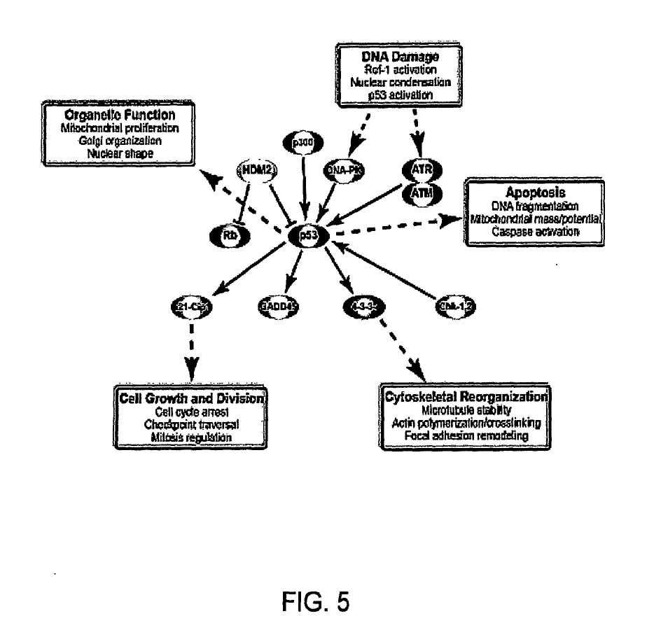

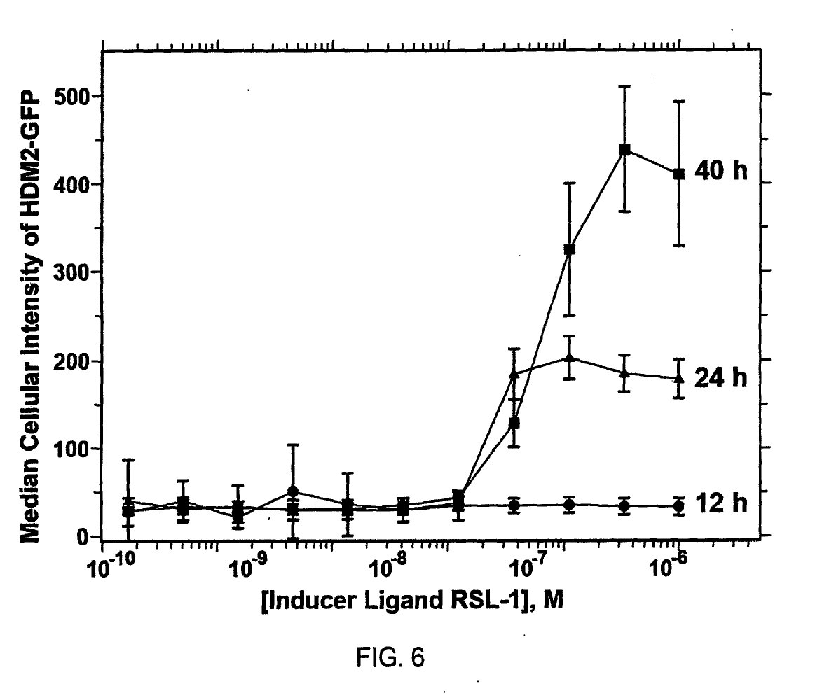

Therefore, described herein is a cell model where different components of the p53 pathway and other cellular processes can be manipulated, so that the expression levels can be regulated to monitor the impact on the downstream cellular events and any off-target and/or compensatory effects. The effects that p53 “knockdown” with RNAi have on the response of A549 cells to multiple anticancer agents have been demonstrated [1]. In this cellular systems biology example, the p53 pathway regulation (FIG. 5) was “manipulated” by controlling the expression level of the p53-modulating protein HDM2 with a gene switch. A profile of the cellular response was built by “measuring” a panel of cellular systems biology biomarkers that included the p53 pathway as well as other possible cellular “systems” responses either downstream (e.g., apoptosis, cell growth and division, cytoskeletal reorganization, and organelle function) or upstream (e.g., DNA damage) (FIG. 5). A single vector RheoSwitch™ (RheoGene; Norristown, Pa.) was used to regulate expression of a fluorescently labeled HDM2 protein in A549 cells using multiple concentrations of inducer molecule. FIG. 6 shows that the expression level of fluorescent HDM2 was dependent on the concentration of the inducer molecule used to treat the cells as well as the total time of induction.

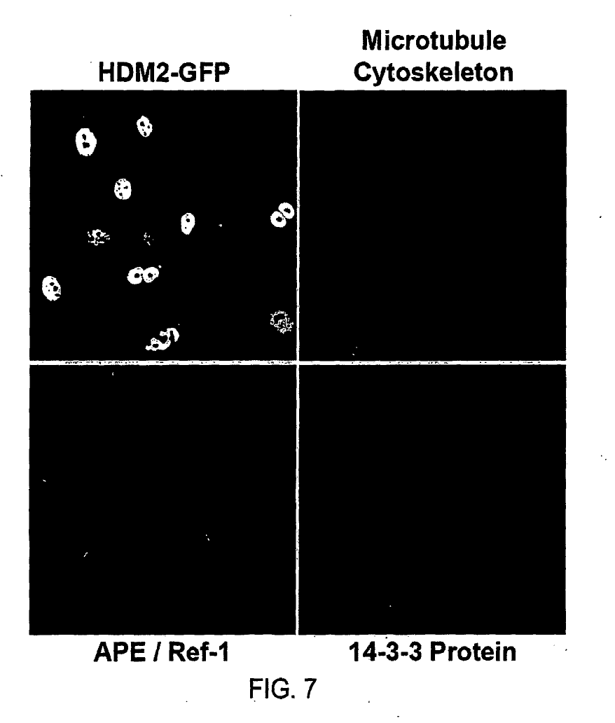

A fixed end point cellular systems biology assay was designed to generate a profile that elucidates the cell system response to up-regulating HDM2 in the absence of any other manipulation. Besides the quantification of HDM2 expression in each cell, Table II shows the additional cellular function biomarkers measured to build the profile. The measurements were chosen to look both at specific down stream activities and more general cellular processes. Furthermore, the biomarkers labeled with the Hoechst 33342 reagent (dna, nu1, nu2, nu3, and nu4) provided redundant feature measurements in both sets of wells because both sets of wells were similarly labeled with Hoechst 33342 reagent. This approach was used to confirm uniform treatments between wells, and was used to build relationships between biomarker measurements even though they were not measured in the same cells. Example images of cells in which cellular systems biology reagents were used to measure and manipulate activity are shown in FIG. 7.

TABLE II
Features measured in the cellular systems biology profile
and their roles in the regulation of cellular systems.

| | Abbreviation | Cellular System |
|---|---|---|
| Measurement | for Heat Map | Regulation |
| Total nuclear intensity and | dna, nu4 | DNA content: |
| variation of Hoechst 33342 | | Cell cycle arrest |
| label | | DNA degradation |
| Projected nuclear area and | nu1, nu2, nu3 | Nuclear condensation or |
| shape defined by Hoechst | | fragmentation |
| 33342 label | | |
| Nuclear intensity of | ph3 | G₂/M cell cycle transition |
| phosphor-histone H3 label | | |
| Cytoplasmic intensity of | tub | Microtubule stability |
| α-tubulin label after | | |
| detergent extraction | | |
| Cytolasmic vinculin label | vin | Focal adhesion remodeling |
| at substrate | | |
| Nuclear/cytoplasm ratio of | p53 | Tumor suppressor involved |
| p53 label | | in DNA damage response. |
| | | HDM2 inhibits p53 upon |
| | | complex formation |
| Nuclear/cytoplasm ratio of | p21 | Tumor suppressor inhibitor |
| p21 label | | of cell cycle progression. |
| | | Active p53 up regulates p21 |
| | | expression |
| Cytoplasmic intensity of | akt | Activated Akt promotes cell |
| phospho-Akt label | | survival by causing the |
| | | inhibition of apoptosis |
| | | targets |
| Nuclear intensity of | ape | APE/Ref-1 is activated |
| APE/Ref-1 label | | upon DNA damage or |
| | | oxidative stress |
| Cytoplasmic level and | x43 | 14-3-3 proteins regulate cell |
| organization of 14-3-3 | | survival, apoptosis, |
| protein | | proliferation, and |
| | | checkpoint control through |
| | | phosphorylation-dependent |
| | | protein-protein interactions |
| Organization and intensity | gol, mit | Organelles such as |
| of Golgi and mitochondrial | | mitochondria play specific |
| organelles | | roles in many cellular |
| | | processes including |
| | | apoptosis |

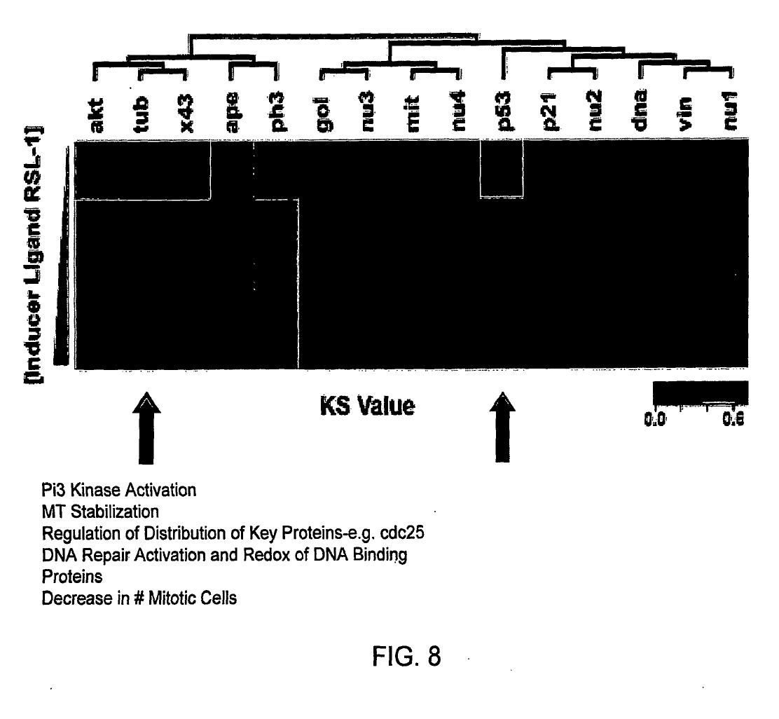

The considerable number of cellular systems biology measurements made after treatment of cells with multiple inducer molecule concentrations produced a large and complex cellular systems biology profile. Automated hierarchical clustering analysis based on Kolmogorov-Smirnov (KS) population dissimilarity analysis provided an ideal first step in deciphering the profiling data [1, 5]. FIG. 8 shows the clustered profiling results in the form of a heat map. Cell population responses that were not significantly different from control populations (e.g., KS<0.2) were encoded in the heat map using blue and black shades. Significant cell population responses induced by HDM2 (e.g., KS>0.2) were encoded in the heat map using yellow and red shades. In the overall response, there were three relatively distinct clusters: high responders (FIG. 8, left); low responders (FIG. 8, center); and intermediate responders (FIG. 8, right).

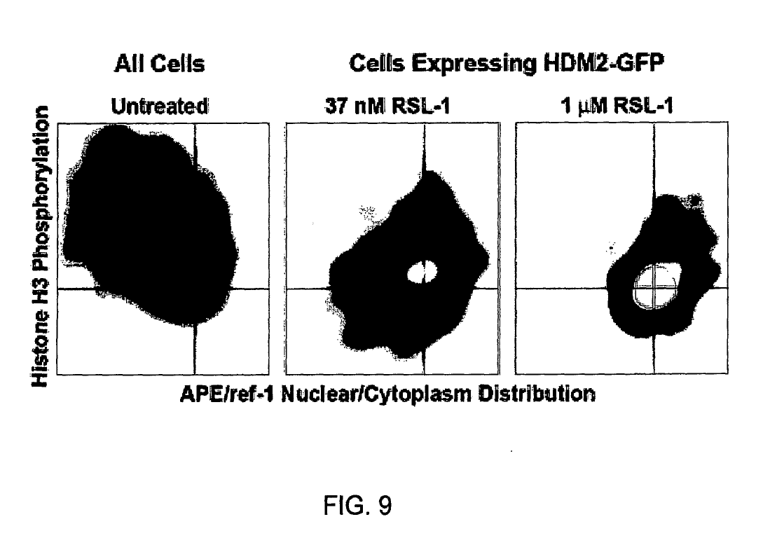

It was expected (FIG. 8; right arrow) that the elevation of HDM2 would decrease the cellular level of p53 which did occur. However, simply raising the cellular concentration of HDM2 about 5× had unexpected effects such as; stabilizing microtubules, increasing PI3 kinase activation, and increasing the level of DNA repair, and redox of DNA binding proteins. To enable the building of connections between the cellular processes measured in the profile, cell maps were used to visualize how two imaging microscopy feature measurements were related in the same cells under different conditions [1, 5]. In this example, the heat map data in FIG. 8 showed that the changes in phospho-histone H3 levels and the intracellular distribution of APE/Ref-1 as a function of HDM2 expression level were similar enough for them to be clustered together. Using the cell maps shown in FIG. 9, the relationship between these two markers of cell cycle regulation and the DNA damage—oxidative stress response was demonstrated. The cell maps show that as the expression level of HDM2 increased in response to RSL-1, the phospho-histone H3 level homogeneously decreased to a lower level (vertical shift down), while the predominant nuclear localization of APE/Ref-1 increased (horizontal shift right), consistent with some common regulatory unit that coordinates the two processes. Thus, this example demonstrates the cellular systems biology approach to build new knowledge on the regulatory connections between cellular processes through the selective Manipulation and measurement of cellular constituents.

### EXAMPLE 2

### A Cellular Systems Biology Model of Huntington's Disease—Manipulation of Mutant Huntingtin Expression Levels

Huntington Disease (HD) is a debilitating and ultimately fatal autosomal dominant disorder of the central nervous system (CNS). HD is the most common inherited neurodegenerative disease with initial manifestation of symptoms occurring in the middle ages of life. Clinical features develop progressively and are characterized by motor dysfunction, cognitive impairment, and psychiatric abnormalities, which lead to death approximately 15-20 years after disease onset. Unfortunately, no effective treatment exists to prevent or even slow HD progression.

HD belongs to a group of neurological diseases characterized by a trinucleotide expansion within the defective gene resulting in the expression of a polyglutamine (polyQ) expanded farm of the encoded protein. The HD protein, huntingtin (Htt), typically causes HD when the glutamine repeat length reaches 36 with disease onset inversely related to repeat length. Htt is ubiquitously expressed and has been implicated in regulating diverse cellular processes including intracellular trafficking, transcriptional regulation, cell signaling, endocytosis, and metabolism. Unlike the wild type form of Htt, the mutant polyQ expanded form of Htt preferentially forms large intracellular aggregates and promotes cytotoxicity primarily in γ-aminobutyric acid-secreting spiny-projection neurons of the striatum. However, the mechanism driving aggregate formation and the toxicity gain of function are presently unknown. Consequently, potential targets for HD drug discovery remain poorly defined, thereby hampering the identification of HD therapeutic agents.

Given the complexity of HD disease and the many cellular processes potentially regulated by Htt, the development of a cellular systems biology model of HD is critical. A cellular systems biology characterization of a model of HD where the expression level of mutant Htt is modulated will provide a phenotypic multiparameter cellular profile of this devastating disease, thereby furthering the understanding of the diverse and complex pathways impacted by mutant huntingtin. Moreover, a cellular systems biology approach will provide a much needed well-validated tool for HD drug discovery. Toward this end, a cellular systems biology model of HD is described in detail below.

In one embodiment, the HD cellular model utilizes a rat phaeochromocytoma PC12 cell line (PC12-HttQ103). These cells represent a well characterized neuronal cell model and have the added advantage that their differentiation status can be acutely regulated. This cell model was engineered to express human Htt exon 1 plus an expanded repeat of 103 glutamines fused to the N-terminus of EGFP (mutant huntingtin-GFP). Expression is driven by an ecdysone inducible promoter such that mutant huntingtin-GFP is only expressed in the presence of the inducer ligand (RSL1) allowing the profiling the cellular systems response to mutant huntingtin expression.

In another embodiment, multiplexed fluorescent based imaging can be combined with imaging microscopy to generate cellular systems biology profiles of HD. Example cellular systems biology profiling features that were measured with and without modulation of Htt expression levels using four fluorescence channels are as follows:

1. Hoechst 33342 (DNA nuclear label) was used to measure:
a. Cell Number
b. Cell Cycle
c. Nuclear size

2. A GFP Htt fusion protein was used to measure:
a. mutant huntingtin-GFP expression levels
b. mutant huntingtin-GFP compartmentalization
c. mutant huntingtin-GFP aggregate formation

3. Toxicity Markers:
a. α-phospho c-Jun
b. α-p53

4. Differentiation
a. α-tubulin for neurite outgrowth determination

To further refine a cellular systems biology profile, up to ten other cellular biomarkers, such as those specified under the additional exemplification can be applied to the analysis.

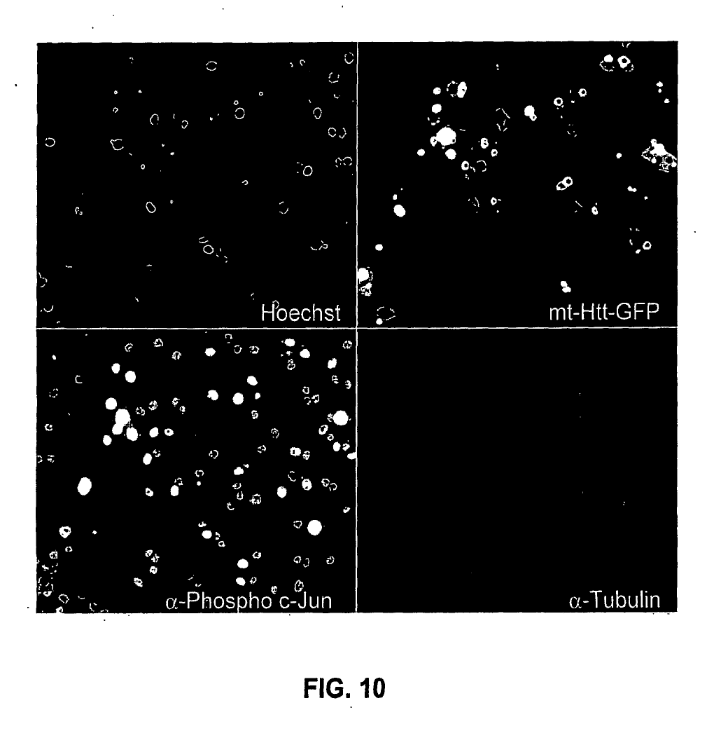

FIG. 10 shows a representative array of multiplexed images generated with the example cell treatment listed above. In this figure, PC12-HttQ103 cells were treated with NGF to promote differentiation and with RSL1 to induce mutant huntingtin-GFP expression. Following fixation and staining, multiplexed images were captured imaging microscopy, and Hoechst 3332 labeled nuclei, mutant huntingtin-GFP expression, phospho c-Jun stained nuclei, and labeled tubulin cells are visualized. To further refine a cellular systems biology profile, up to ten other cellular biomarkers, such as those specified under the additional exemplification can be applied to the analysis.

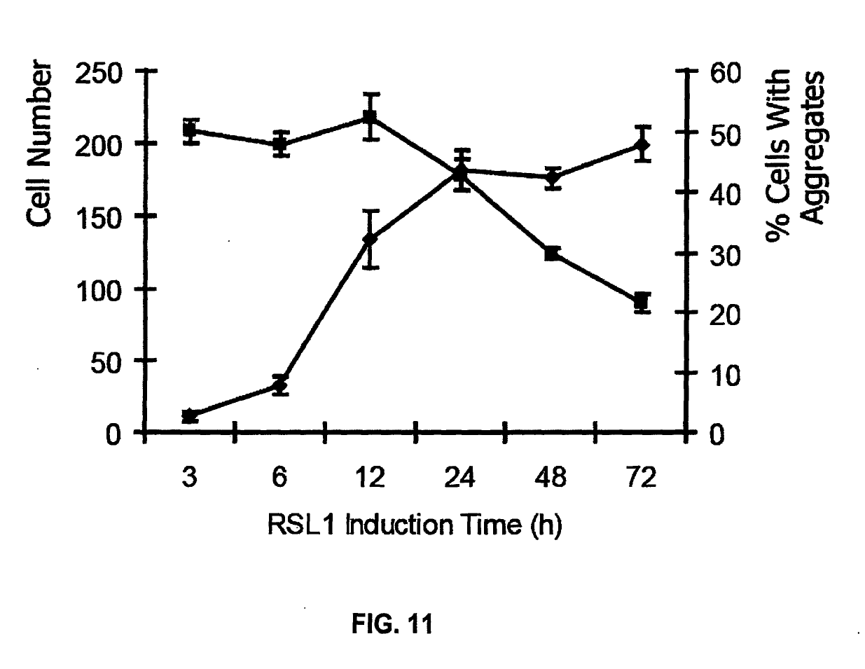

A cellular systems biology approach was used to profile the effect of mutant huntingtin-GFP expression on several example parameters. First assessed was whether mutant huntingtin-GFP expression altered cell loss and whether changes in cell numbers correlate with aggregate formation. Hoechst 33342 labeled nuclei were utilized to determine cell numbers and mutant huntingtin-GFP aggregates were measured using standard imaging algorithms. As shown in FIG. 11, cell number decreased as a function of RSL1 treatment time and this cell loss correlated with an increase in mutant huntingtin-GFP aggregate formation. These two changes are well established characteristics of HD neurons thereby validating these features as useful cellular systems biology profiling features. As shown in FIG. 11, only 50% of the cells formed aggregates. With this approach, changes can be profiled independently within these two distinct aggregate producing and non-aggregate producing cellular populations (see below). To further refine a cellular systems biology profile, up to ten other cellular biomarkers, such as those specified under the additional exemplification can be applied to the analysis.

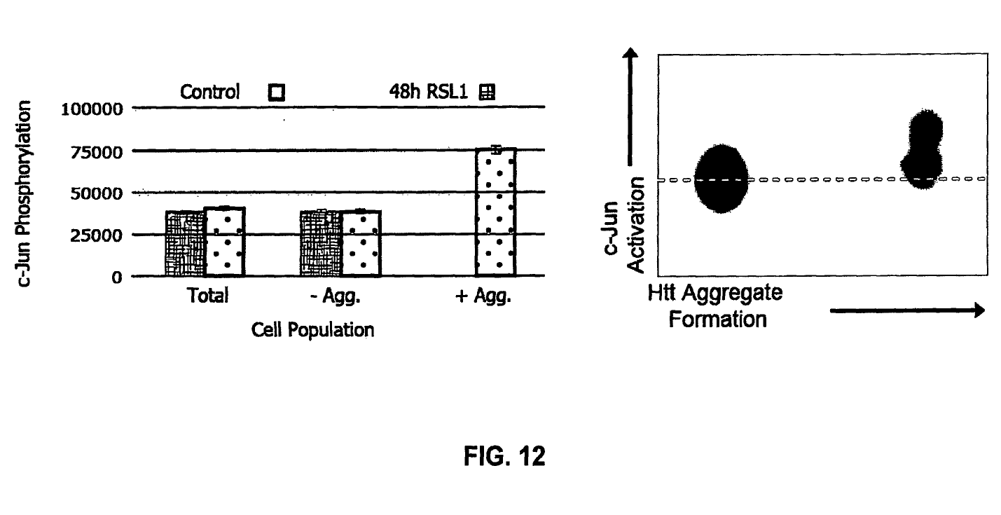

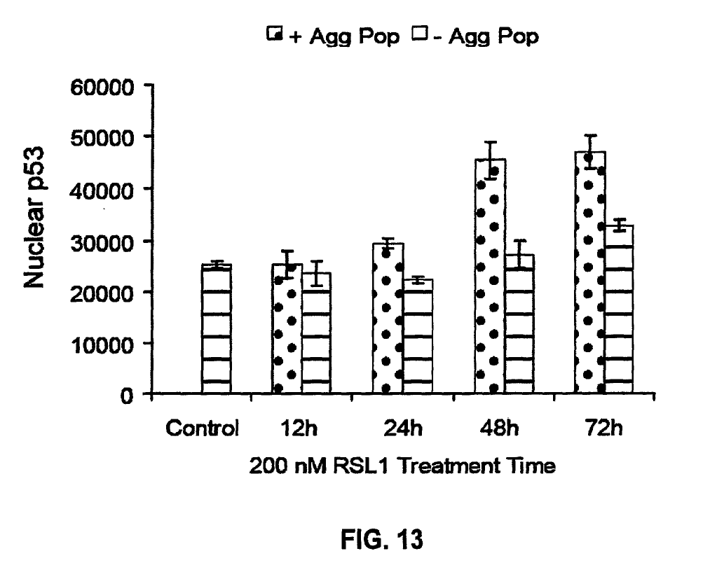

Two other example cellular systems biology features were also demonstrated using the cellular model of HD. Cells were induced with RSL1 for the indicated treatment times and were labeled for either c-jun phosphorylation level or the activation of p53. Nuclear intensity levels for each of these markers were determined in the total cell population, the aggregate-producing population (+Agg), or the non-aggregate-forming cells (−Agg). As demonstrated in FIG. 12 (phospho c-Jun) and FIG. 13 (p53) both toxicity markers were selectively elevated in the aggregate-producing population of cells further correlating toxicity with aggregate production. To further refine a cellular systems biology profile, up to ten other cellular biomarkers, such as those specified under the additional exemplification can be applied to the analysis.

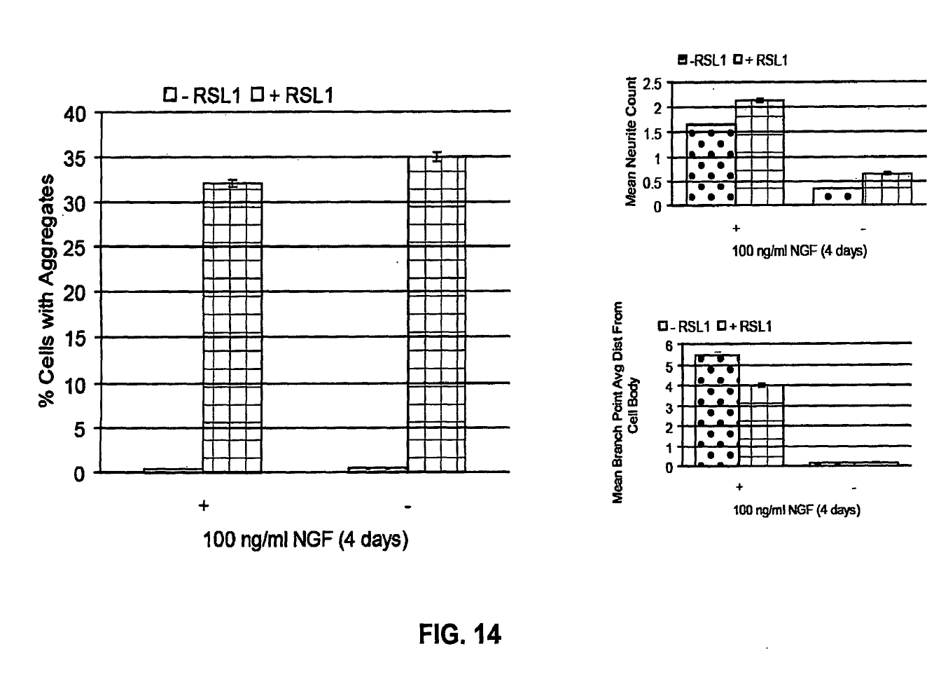

Yet another example cellular systems biology profiling feature was demonstrated to determine whether the differentiation state of the cells impacted aggregate formation and whether mutant huntingtin-GFP expression affected neurite outgrowth (FIG. 14). As illustrated in panel A, treatment with NGF for four days had little impact on aggregate formation. NGF did increase mean neurite count (panel B) and mean branch point distance from the cell body (panel C). The expression of mutant huntingtin-GFP (+RSL1) resulted in a modest increase in mean neurite count but a slight reduction in mean branch point distance from the cell body. To further refine a cellular systems biology profile, up to ten other cellular biomarkers, such as those specified under the additional exemplification can be applied to the analysis.

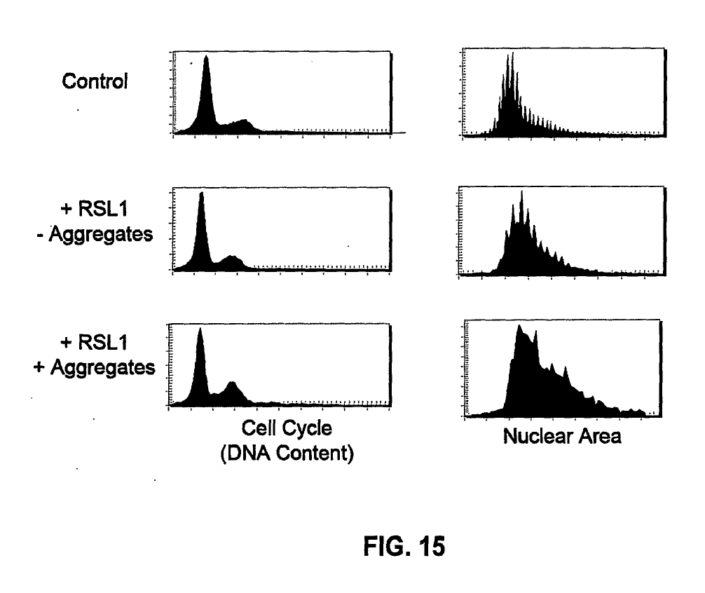

Another example cellular systems biology profiling feature was used to correlate aggregate formation with changes in the cell cycle and nuclear area, a measure of cell health (FIG. 15). As illustrated in FIG. 15A, the cellular distribution for DNA content shifted slightly to 4N DNA content for the aggregate forming cell population with no change observed for the non-aggregate producing cells. Also, the cellular distribution profile for nuclear area exhibited a rightward shift selectively for the aggregate-forming cells. To further refine a cellular systems biology profile, up to ten other cellular biomarkers, such as those specified under the additional exemplification can be applied to the analysis.

The cellular systems biology profile using the Huntington disease cellular model can also comprise further additional cellular systems biology feature measurements after the cells have been manipulated and include those described in the additional exemplification below.

### EXAMPLE 3

### Cellular Systems Biology Response Profile to Huntingtin Protein Aggregation Formation in the Presence of Microtubule Modulating Drugs

To test if there was an interrelationship between the modulation of the microtubule cytoskeleton and the formation of huntingtin protein (Htt) aggregates, cells were induced to produce fluorescently labeled huntingtin protein (GFP-Htt). Soon thereafter, the same Cells were treated with a set of compounds including several microtubule modulators. Cellular systems biology profiling can be used to define the interrelationships between several cellular features including microtubule cytoskeletal stability, cell cycle regulation, GFP-Htt aggregate formation, and nuclear morphology.

In one embodiment, 7.5×10⁺⁶ PC12T cells were plated into T25 tissue culture flasks and panasterone A was added to a final concentration of 5 μM. After an overnight incubation, the cells were trypsinized and replated onto collagen I coated 384-well microplates at a density of 10000 cells per well in medium containing 1 μM panasterone A (24 h after the first panasterone A treatment began). Cells were treated for 26 h with 14 compounds at 12 concentrations each ranging from <1 nM to 50 μM. At the end of the incubation period, cells were fixed with 3.7% formaldehyde, permeabilized with 0.5% Triton X-100 detergent, and labeled with primary-secondary antibody pairs to measure microtubule stability (α-tubulin) and mitotic events (phospho-histone 113). Cells were also labeled with Hoechst 33342 (5 μg/ml) to provide measurements of cell cycle regulation (DNA content), changes in cell number, and multiple nuclear morphology measurements including chromatin condensation and nuclear size and shape.

To build a cellular systems biology profile of the cellular response to GFP-Htt aggregation and compound treatment, the following example cellular systems biology response profile features can be calculated from responses of cell populations either expressing GFP-Htt or not expressing GFP-Htt that can be separated mathematically from each other and analyzed separately.

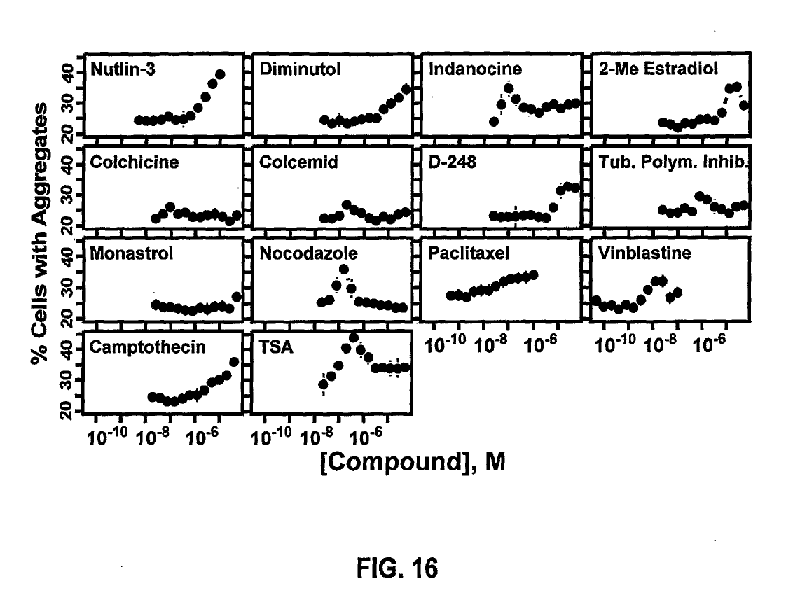

As one example of a cellular systems biology profiling feature, FIG. 16 shows the effects that chemical compounds had on the formation of GFP-Htt aggregates in PC12T cells. Compounds either had no effect on GFP-Htt aggregate formation at multiple concentrations, or either induced or inhibited aggregate formation at one or more concentrations. Both microtubule stabilizers and destabilizers modulated GFP-Htt aggregate formation.

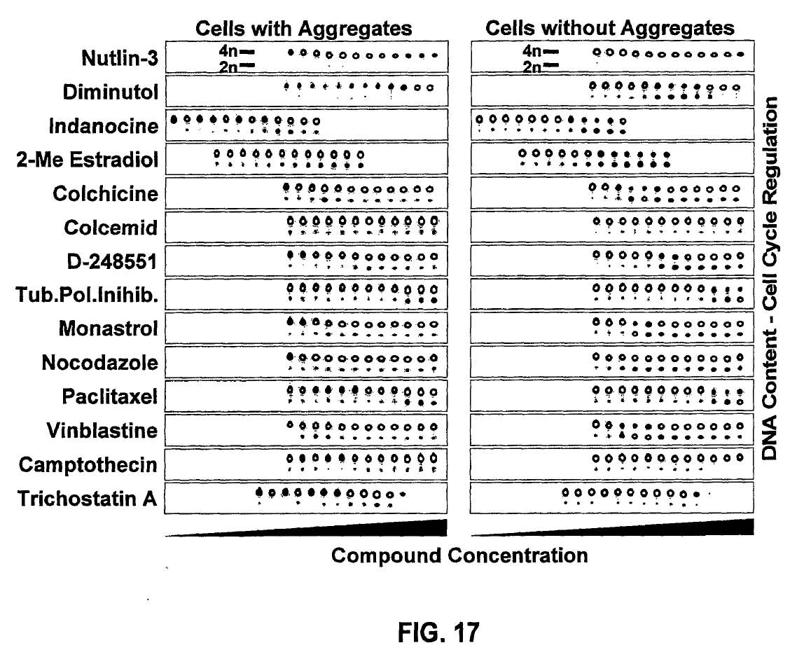

As another example of a cellular systems biology profiling feature, FIG. 17 shows that compound induced modulation of cell cycle regulation (e.g., 2 n/4 n distribution ratio) was itself modulated in the presence of GFP-Htt aggregates. For example, cells with aggregates were less responsive to the compound nocodazole than were cells with out aggregates.

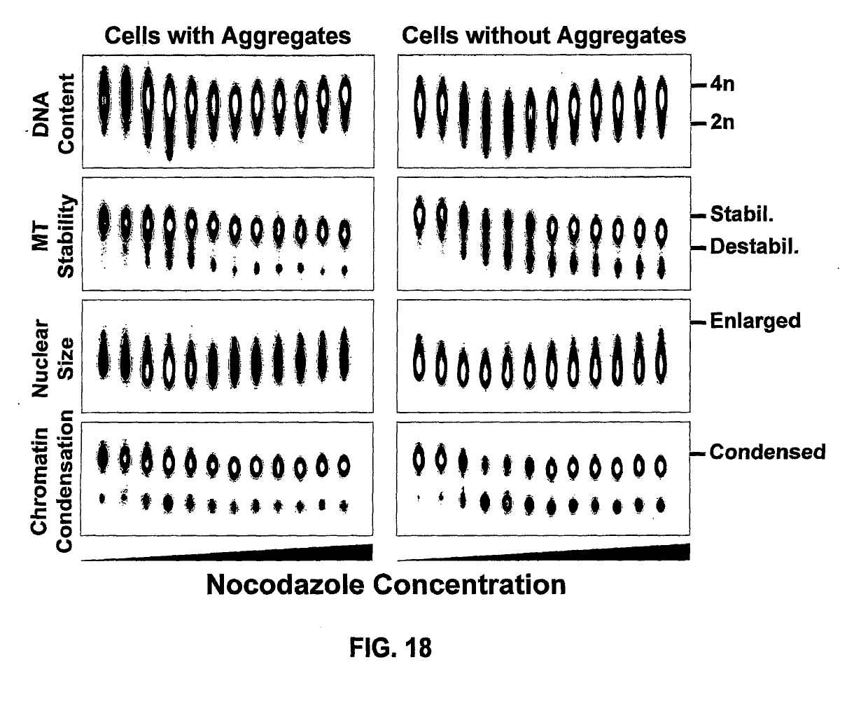

Yet another example cellular systems biology profiling features is shown in FIG. 18. The effects of nocodazole on DNA content, microtubule stability, nuclear size, and chromatin condensation are shown in cells either with GFP-Htt aggregates or in cells without aggregates. Nocodazole-induced cell cycle regulation and nuclear morphological changes were partially inhibited in cells containing aggregates. Furthermore, cells with aggregates exhibited greater sensitivity to the microtubule destabilizing activity of nocodazole than cells without aggregates.

To add to the cellular systems biology profile using the Huntington disease cellular model, further additional cellular systems biology feature measurements are included in the in the profile after the cells have been manipulated and include those described in the additional exemplification below.

Thus, the manipulation of Htt expression levels coupled with cellular systems biology profiling of the cellular response to compound treatment enabled the discovery of molecular interrelationships at the systems level of a cellular model of disease.

### EXAMPLE 4

### Cellular Model of Cancer Using a Knockdown of a Cell Cycle Regulatory Protein in Conjunction with a Cellular Systems Biology Profiling Approach

Aberrant control of the cell cycle is one of the hallmarks of cancer. One possible cellular model of cancer involves the deliberate down-modulation (e.g., knockdown) of a cell cycle regulatory protein, hKIS, coupled with a cellular systems biology profile of the effects of the knockdown on human tumor cells, with and without compound treatment.

In one embodiment, double stranded short inhibitory RNA molecules (siRNAs) are used to modulate the expression level of artificially over expressed hKIS, a kinase known to phosphorylate p27, another cell cycle regulatory protein in a tumor cell model system. For example, a human osteosarcoma cell line, U2OS can co-transfected using an electroporation method known in the art with 0.1-5.0 μg of DNA vector encoding fluorescently labeled hKIS (e.g., TagGFP-hKIS) or with DNA vector encoding an epitope labeled hKIS (e.g., HAHA-hKIS) plus 10-1000 nM hKIS siRNA (e.g., Ambion, Inc. and Dharmacon, Inc., commercially available) or scrambled siRNA (e.g., Ambion, Inc.) and allowed to recover overnight. Cells can be transferred to 384-well microplates and incubated an additional period (1-24 h) before fixation with a formaldehyde solution (3.7%). Cellular nuclei can be labeled with Hoechst 33342 to enable the measurement of other cellular systems biology profiling features such as DNA content, chromatin condensation, and other nuclear Morphology features.

Further additional cellular systems biology feature measurements are included in the in the profile after the cells have been manipulated and include those described in the additional exemplification below.

Moreover, cellular systems biology profiling of cellular models modulated by the addition of chemical compounds or other treatments for various periods of time provide additional modes of cellular systems manipulation.

To quantify the knockdown of the artificially over expressed hKIS, high-content analysis was performed. Expression levels of TagGFP-hKIS and HAHA-hKIS relative to the scrambled siRNA controls were determined. For cells transfected with the TagGFP-hKIS vector, the following percent remaining expression levels were measured: Ambion #1208—6.7%; Ambion #1398—6.8%; Dharmacon #2—6.7%; and Dharmacon #3—6.8%. For cells transfected with the HAHA-hKIS vector, the following percent remaining expression levels were measured: Ambion #1208—7.7%; Ambion #1398—9.1%; Dharmacon #2—6.5%; and Dharmacon #3—5.6%. Thus, all four siRNAs tested knocked down the expression level of cytomegalovirus (CMV) promoter regulated hKIS fusion proteins to a level that was <10% of control, confirming the method for further analysis.

To determine the effects of hKIS knockdown on a cancer cellular systems biology model, an expression vector encoding HA-tagged p27 and siRNAs directed against hKIS were cotransfected using the protocol above except that 2.0 μg of HA-tagged p27 vector (wild type and two mutant isotypes) was cotransfected with and without 500 nM each of hKIS siRNAs (Dharmacon #2 and #3). As part of the cellular systems biology profile, the distribution of the overexpressed p27 isoforms between the nucleus and the cytoplasm was measured as a function of the level of p27 over expression. To further refine a cellular systems biology profile, up to ten other cellular biomarkers, such as those specified under the additional exemplification can be applied to the analysis.

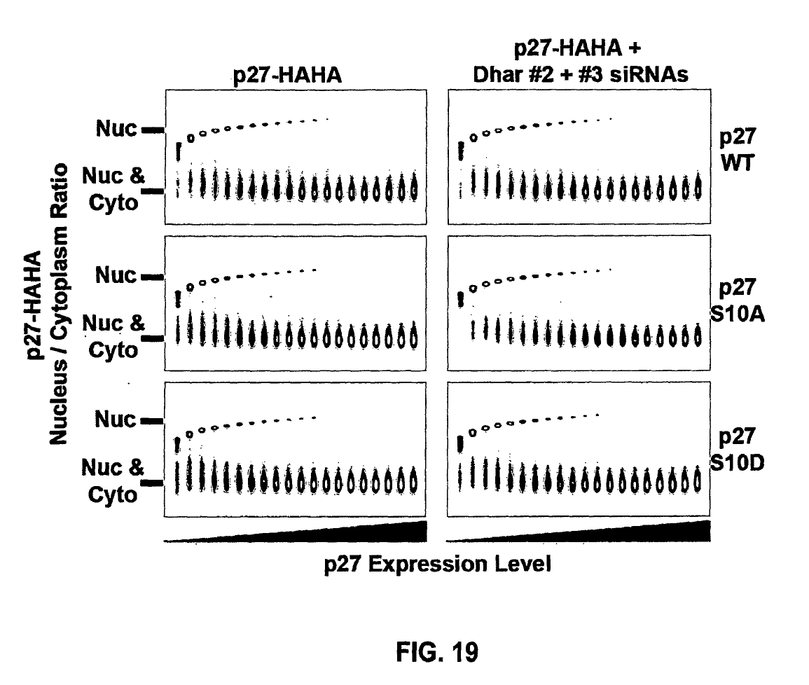

FIG. 19 shows the resulting cellular systems biology profiling distribution maps of the effects that hKIS protein knockdown has on the intracellular distribution of over expressed wild type and mutant p27 isoforms. The p27 distribution in U2OS cells cotransfected with HA-tagged p27 isoforms and hKIS siRNAs was dependent on the p27 expression level, but independent of the isoforms of the p27 being over expressed and also independent of the knockdown of endogenous hKIS using siRNAs. To further refine a cellular systems biology profile, up to ten other cellular biomarkers, such as those specified under the additional exemplification can be applied to the analysis.

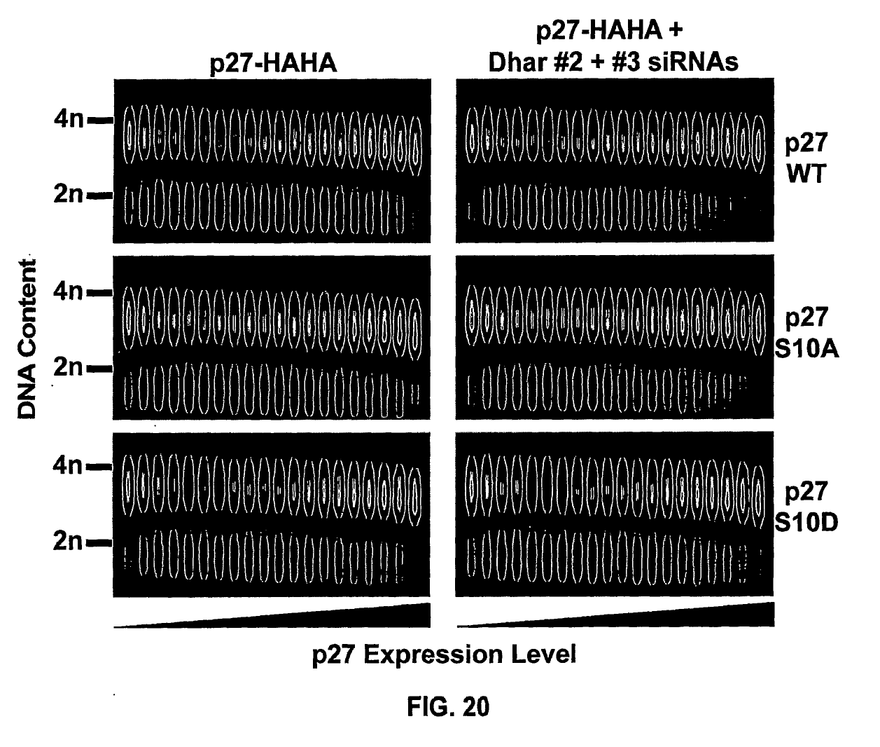

FIG. 20 shows the resulting cellular systems biology profiling distribution maps of the effects that hKIS protein knockdown has on the regulation of the cell cycle, specifically the DNA content, in cells with over expressed wild type and mutant p27 isoforms. The DNA content in U2OS cells cotransfected with HA-tagged p27 isoforms and hKIS siRNAs was dependent on the p27 expression level, but independent of the isoforms of the p27 being over expressed and also independent of the knockdown of endogenous hKIS using siRNAs. To further refine a cellular systems biology profile, up to ten other cellular biomarkers, such as those specified under the additional exemplification can be applied to the analysis.

Cellular systems biology profiling of cellular models modulated by the addition of chemical compounds or other treatments for various periods of time provide additional modes of cellular systems manipulation.

Thus, cellular manipulation through protein over expression, reduction of protein expression levels via siRNA activity, or a combination of both, coupled with cellular systems biology profiling provides the basis of relevant cellular models of disease.

### ADDITIONAL EXEMPLIFICATION

In any one of the examples described above, further additional cellular systems biology feature measurements can be included in the in the profile after the cells have been manipulated and include, for example and without limitation:

Temporal and spatial measurement of specific molecular processes using fluorescent protein biosensors that have been introduced into the cells
Kinase activation
Protease activation
Protein-protein interactions

Organelle function
Mitochondrial membrane potential and mass
Lysosomal mass
Nuclear shape and size
Peroxisomal mass
Golgi organization

Cytoskeletal structure
Microtubule cytoskeleton stability
Actin cytoskeleton assembly state
Intermediate filament cytoskeleton assembly state
Cytoplasmic level and organization of 14-3-3 protein
Cytoplasmic vinculin at the substrate

Transcription factor activation
NF-κB
Stat proteins
Oct-1
ATF proteins
NFAT proteins
SMAD proteins
Phospho-CREB
HIF-1 alpha
FOXO3a

Cell cycle regulation
histone H3 phosphorylation level
incorporation of BrdU into newly synthesized DNA
the formation of micronuclei

Regulation of stress kinase and other kinase activities
phosphorylation level of c-jun
activation of ERK-MAPK
activation of p38
activation of INK
activation of protein kinase A
activation of protein kinase C
activation of Akt
activation of PI3K

Morphological phenotypic changes
Cell spreading
Cell attachment
Cell motility
Cell count
Differentiation (e.g., neurite outgrowth of neuronal cells)

Response to DNA damage
p53 activation through nuclear translocation
p53 activation through phosphorylation
p21 activation
phosphorylation level of histone H2A.X

Induction of apoptosis
cytochrome c loss from mitochondria
extracellular annexin V binding
cleavage of PARP
activation of caspase activities

### REFERENCES

Abraham, V. C., D. L. Taylor, and J. R. Haskins. 2004a. High content screening applied to large-scale cell biology. Trends In Biotechnology. 22:15-22.

Aloy, P., and R. B. Russell. 2005. Structure-based systems biology: a zoom lens for the cell. FEBS Lett. 579:1854-8.

Araujo, R. P., and L. A. Liotta. 2006. A control theoretic paradigm for cell signaling networks: a simple complexity for a sensitive robustness. Curr Opin Chem Biol.

Bugrim, A., T. Nikolskaya, and Y. Nikolsky. 2004. Early prediction of drug metabolism and toxicity: systems biology approach and modeling. Drug Discov Today. 9:127-35.

Butcher, E. C., E. L. Berg, and E. J. Kunkel. 2004. Systems biology in drug discovery. Nat Biotechnol. 22:1253-9.

Collins, F. S., E. D. Green, A. E. Guttmacher, and M. S. Guyer. 2003a. A vision for the future of genomics research. Nature. 422:835-47.

Collins, F. S., M. Morgan, and A. Patrinos. 2003b. The human genome project: lessons from large-scale biology. Science. 300:286-90.

Costa, F. F. 2005. Non-coding RNAs: new players in eukaryotic biology. Gene. 357:83-94.

Cummins, J. M., Y. He, R. J. Leary, R. Pagliarini, L. A. Diaz, Jr., T. Sjoblom, O. Barad, Z. Bentwich, A. E. Szafranska, E. Labourier, C. K. Raymond, B. S. Roberts, H. Juhl, K. W. Kinzler, B. Vogelstein, and V. E. Velculescu. 2006. The colorectal microRNAome. Proc Natl Acad Sci U S A. 103:3687-92.

Danna, E. A., and G. P. Nolan. 2006. Transcending the biomarker mindset: deciphering disease mechanisms at the single cell level. Curr Opin Chem Biol.

Giuliano,. K. A., W. S. Cheung, D. P. Curran, B. W. Day, A. J. Kassick, J. S. Lazo, S. G. Nelson, Y. Shin, and D. L. Taylor. 2005. Systems cell biology knowledge created from high content screening. Assay Drug Dev Technol. 3:501-14.

Giuliano, K. A., R. L. DeBiasio, R. T. Dunlay, A. Gough, J. M. Volosky, J. Zock, G. N. Paviakis, and D. L. Taylor. 1997. High-content screening: A new approach to easing key bottlenecks in the drug discovery process. Journal of Biomolecular Screening. 2:249-259.

Giuliano, K. A., J. R. Haskins, and D. L. Taylor. 2003. Advances in high content screening for drug discovery. ASSAY and Drug Development Technologies. 1:565-577.

Giuliano, K. A., D. L. Taylor, and A. S. Waggoner. 2006a. Reagents to measure and manipulate cell functions. In High Content Screening: A Powerful Approach to Systems Cell Biology and Drug Discovery. Vol. 356. D. L. Taylor, Haskins, J. R., and Giuliano, K. A., editor. Humana Press, Totowa, NJ. 141-163.

Giuliano, K. A., P. A. Johnston, A. Gough, and D. L. Taylor. 2006b. Systems cell biology based on high-content screening. Methods Enzymol. 414:601-619.

Glocker, M. O., R. Guthke, J. Kekow, and H. J. Thiesen. 2006. Rheumatoid arthritis, a complex multifactorial disease: on the way toward individualized medicine. Med Res Rev. 26:63-87.

Gough, A. H., and P. A. Johnston. 2006. Requirements, features and performance of high content screening platforms In High Content Screening: A Powerful Approach to Systems Cell Biology and Drug Discovery. D. L. Taylor, Haskins, J. R., and Giuliano, K. A., editor. Humana Press, Totowa, N.Y. (in press).

Hood, L., and R. M. Perlmutter. 2004. The impact of systems approaches on biological problems. in drug discovery. Nat Biotechnol. 22:1215-7.

Hopkins, A. L., and C. R. Groom. 2002. Opinion: The druggable genome. Nat Rev Drug Discov. 1:727-30.

Huttenhofer, A., P. Schattner, and N. Polacek. 2005. Non-coding RNAs: hope or hype? Trends Genet. 21:289-97.

Jain, S., N. W. Wood, and D. G. Healy. 2005. Molecular genetic pathways in Parkinson's disease: a review. Clin Sci (Loud). 109:355-64.

Janes, K. A., and D. A. Lauffenburger. 2006. A biological approach to computational models of proteomic networks. Curr Opin Chem Biol.

Melnick, J. S., J. Janes, S. Kim, J. Y. Chang, D. G. Sipes, D. Gunderson, L. James, J. T. Matzen, M. E. Garcia, T. L. Hood, R. Beigi, G. Xia, R. A. Harig, H. Asatryan, S. F. Yan, Y. Zhou, X. J. Gu, A. Saadat, V. Zhou, F. J. King, C. M. Shaw, A. I. Su, R. Downs, N. S. Gray, P. G. Schultz, M. Warmuth, and J. S. Caldwell. 2006. An efficient rapid system for profiling the cellular activities of molecular libraries. Proc Natl Acad Sci U S A. 103:3153-8.

Nadeau, J. H., L. C. Burrage, J. Restivo, Y. H. Pao, G. Churchill, and B. D. Hoit. 2003. Pleiotropy, homeostasis, and functional networks based on assays of cardiovascular traits in genetically randomized populations. Genome Res. 13:2082-91.

O'Brien, P. J., and J. R. Haskins. 2006. In vitro cytotoxicity assessement. In High Content Screening: A Powerful Approach to Systems Cell Biology and Drug Discovery. D. L. Taylor, Haskins, J. R., and Giuliano, K. A., editor. Humana Press, Totowa, N.J. (in press).

O'Brien, P. J., W. Irwin, D. Diaz, E. Howard-Cofield, C. M. Krejsa, M. R. Slaughter, B. Gao, N. Kaludercic, A. Angeline, P. Bernardi, P. Brain, and C. Hougham. 2006. High concordance of drug-induced human hepatotoxicity with in vitro cytotoxicity measured in a novel cell-based model using high content screening. Archives of Toxicology. 80:5 80-604.

Phillips, K. A., and S. L. Van Bebber. 2005. Measuring the value of pharmacogenomics. Nat Rev Drug Discov. 4:500-9.

Taylor, D. L. 2006a. Insights into High Content Screening: Past, Present and Future. In High Content Screening: A Powerful Approach to Systems Cell Biology and Drug Discovery. D. L. Taylor, Haskins, J. R., and Giuliano, K. A., editor. Humana Press, Totowa, N.J. 3-18.

Taylor, D. L., and K. A. Giuliano. 2005. Multiplexed high content screening assays create a systems cell biology approach to drug discovery. Drug Discovery Today: Technologies. 2:149-154.

Taylor, D. L., Haskins, J. R., and Giuliano, K. A. 2006b. High Content Screening: A Powerful Approach to Systems Cell Biology and Drug Discovery. In Methods in Molecular Biology. Vol. 356. J. Walker, editor. Humana Press, Totowa, N.J. 444.

Tuomisto, T. T., B. R. Binder, and S. Yla-Herttuala. 2005. Genetics, genomics and proteomics in atherosclerosis research. Aim Med. 37:323-32.

Volinia, S., G. A. Calin, C. G. Liu, S. Ambs, A. Cimmino, F. Petrocca, R. Visone, M. Iorio, C. Roldo, M. Ferracin, R. L. Prueitt, N. Yanaihara, G. Lanza, A. Scarpa, A. Vecchione, M. Negrini, C. C. Harris, and C. M. Croce. 2006. A microRNA expression signature of human solid tumors defines cancer gene targets. Proc Natl Acad Sci U S A. 103:2257-61.

Westerhoff, H. V., and B. O. Palsson. 2004. The evolution of molecular biology into systems biology. Nat Biotechnol. 22:1249-52.

Yang, Y., E. A. Blomme, and J. F. Waring. 2004. Toxicogenomics in drug discovery: from preclinical studies to clinical trials. Chem Biol Interact. 150:71-85.

1. Giuliano, K. A., Y. T. Chen, and D. L. Taylor, High-content screening with siRNA optimizes a cell biological approach to drug discovery: Defining the role of p53 activation in the cellular response to anticancer drugs. J. Biomol. Screening, 2004. 9(7): p. 557-568.

2. Blagosklonny, M. V. and A. B. Pardee, Exploiting cancer cell cycling for selective protection of normal cells. Cancer. Res, 2001. 61(11): p. 4301-5.

3. Borbe, R., J. Rieger, and M. Weller, Failure of taxol-based combination chemotherapy for malignant glioma cannot be overcome by G2/M checkpoint abrogators or altering the p53 status. Cancer Chemother Pharmacol, 1999. 44(3): p. 217-27.

4. Osaki, S., et al., Alteration of drug chemosensitivity caused by the adenovirus-mediated transfer of the wild-type p53 gene in human lung cancer cells. Cancer Gene Ther, 2000. 7(2): p. 300-7.

5. Giuliano, K. A., et al., Systems cell biology knowledge created from high content screening. Assay Drug Dev Technol, 2005. 3(5): p. 501-14.

6. Giuliano, K. A., D. L. Taylor, and A. S. Waggoner, Reagents to measure and manipulate cell functions, in High Content Screening: A Powerful Approach to Systems Cell Biology and Drug Discovery, D. L. Taylor, Haskins, J. R., and Giuliano, K. A., Editor. 2006, Humana Press: Totowa, N.J. p. 141-163.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A method for profiling a disease state, comprising:
a) obtaining one or more cells associated with a disease state;
b) labeling the one or more cells associated with a disease state with a panel of fluorescently labeled reagents, thereby producing one or more fluorescently labeled cells, wherein each fluorescently labeled reagent is specific for a biomarker, the panel of fluorescently labeled reagents detects at least five different biomarkers, and the detection of a biomarker provides a read-out of one or more features of the one or more cells;
c) imaging the one or more fluorescently labeled cells with at least one optical mode, wherein the imaging produces a first set of data; and
d) analyzing the first set of data to read-out the features of the five or more biomarkers, wherein the combination of the features of the five or more biomarkers generates a cellular systems biology profile of the one or more cells associated with a disease state,
thereby profiling the disease state.

2. The method of claim 1, wherein the one or more cells associated with a disease state are one or more cells isolated from a diseased tissue, or one or more cells manipulated to exhibit one or more disease phenotypes.

3. The method of claim 1, wherein one or more molecules selected from the group consisting of: DNA, RNA, protein, aptamer, peptide tag, carbohydrate, lipid, and a combination thereof, are introduced into the one or more cells associated with a disease state.

4. The method of claim 3, wherein the one or more molecules are conditionally expressed in the one or more cells.

5. The method of claim 4, further comprising generating a cellular systems biology profile of the one or more cells before conditional expression of the one or more molecules, and generating a cellular systems biology profile of the one or more cells after conditional expression of the one or more molecules, wherein the cellular systems biology profiles of the one or more cells before and after conditional expression of the one or more molecules are compared.

6. A method for assessing the effect of an agent on one or more cells associated with a disease state, comprising:
a) contacting the one or more cells associated with a disease state with an agent, thereby producing one or more agent-treated cells
b) labeling the one or more agent-treated cells with a panel of fluorescently labeled reagents, thereby producing one or more fluorescently labeled agent-treated cells, wherein each fluorescently labeled reagent is specific for a biomarker, the panel of fluorescently labeled reagents detects at least five different biomarkers, and the detection of a biomarker provides a read-out of one or more features of the one or more agent-treated cells;
c) imaging the one or more fluorescently labeled agent-treated cells with at least one optical mode, wherein the imaging produces a second set of data;
d) analyzing the second set of data to read-out the features of the five or more biomarkers, wherein the combination of the features of the five or more biomarkers generates a cellular systems biology profile of the one or more agent-treated cells; and
e) comparing the cellular systems biology profile of the one or more agent-treated cells with a control,
thereby assessing the effect of the agent on the one or more cells associated with a disease state.

7. (canceled)

8. (canceled)

9. The method of claim 1, wherein the one or more cells associated with a disease state are one or more cells associated with a cancer, a neurological disease, a metabolic disease, an immunity-related disease, or a combination thereof.

10. (canceled)

11. The method of claim 2, wherein the one or more cells associated with disease state are one or more cells manipulated to express an increased level of p53 as compared to normal cells, wherein the disease phenotype is cancer.

12. (canceled)

13. (canceled)

14. The method of claim 2, wherein the one or more cells associated with a disease state are one or more cells are manipulated to express a mutated Huntingtin protein, wherein the disease phenotype is Huntington's Disease.

15. The method of claim 1, wherein at least the steps of c) and d) are automated.

16. The method of claim 1, further comprising: obtaining cells associated with two or more disease states; generating a cellular systems biology profile of the cells associated with a first disease state; generating a cellular systems biology profile of the cells associated with a second disease state, and comparing the cellular systems biology profiles associated with the first disease state and second disease state.

17. (canceled)

18. (canceled)

19. The method of claim 1, wherein two or more samples of cells associated with a disease state are obtained from an animal at two or more time points, wherein at least one sample of cells is obtained from each time point, the method further comprising: profiling the disease state of the sample of cells obtained from each time point, thereby generating a cellular systems biology profile of each sample of cells obtained from each time point; and comparing the cellular systems biology profiles of the one or more cells obtained from each time point.

20. The method of claim 1, wherein the panel of fluorescently labeled reagents is selected from the group consisting of fluorescently labeled antibodies, fluorescently labeled peptides, fluorescently labeled polypeptides, fluorescent protein biosensors, fluorescently labeled aptamers, fluorescently labeled nucleic acid probes, fluorescently labeled chemicals, fluorescent chemicals, and combinations thereof.

21. (canceled)

22. The method of claim 1, wherein the panel of fluorescently labeled reagents indicate the presence, amount, location, activity, distribution, or combination thereof, of the biomarkers in the one or more fluorescently labeled cells.

23-26. (canceled)

27. The method of claim 1, wherein the cellular systems biology profile is stored in a database for reference, thereby providing a reference cellular systems biology profile in a database, the method further comprising comparing the reference cellular systems biology profile with a cellular systems biology profile of a test sample, the method comprising profiling a test sample of one or more cells, comprising:
a) labeling the one or more cells with a panel of fluorescently labeled reagents, thereby producing one or more fluorescently labeled cells, wherein each fluorescently labeled reagent is specific for a biomarker, the panel of fluorescently labeled reagents detects at least five different biomarkers, and the detection of a biomarker provides a read-out of one or more features of the one or more cells;
b) imaging the one or more fluorescently labeled cells with at least one optical mode, wherein the imaging produces a first second of data;
c) analyzing the second set of data to read-out the features of the five or more biomarkers, wherein the combination of the features of the five or more biomarkers generates a cellular systems biology profile of the test sample of one or more cells; and
d) comparing the cellular systems biology profile of the test sample of one or more cells with the reference cellular systems biology profile,
thereby comparing the reference cellular systems biology profile with the cellular systems biology profile of a test sample.

28. (canceled)

29. The method of claim 1, wherein two or more cells associated with a disease state are located at two or more positions on a plate, and the read-out of the features of the five or more biomarkers is generated from the two or more cells located at two or more positions on the plate, wherein at least one biomarker is the same biomarker in the two or more cells located at two or more positions on the plate.

30. A method for profiling a cancer, comprising:
a) obtaining one or more cells associated with a cancer,
b) labeling the one or more cells associated with a cancer with a panel of fluorescently labeled reagents, thereby producing one or more fluorescently labeled cells, wherein each fluorescently labeled reagent is specific for a biomarker, and wherein the panel of fluorescently labeled reagents detects eleven or more biomarkers, and wherein the detection of a biomarker is a read-out of one or more features of a cellular systems biology profile, and wherein the one or more features can be the same or different for each biomarker detected;
c) imaging the one or more fluorescently labeled cells with at least one optical mode, wherein the imaging produces a first set of data; and
d) analyzing the first set of data to read-out the eleven or more biomarkers, wherein the combination of the eleven or more biomarkers generates a cellular systems biology profile of the one or more cells associated with a disease state,
thereby profiling the cancer.

31. The method of claim 30, wherein the one or more cells associated with a cancer are isolated from a cancerous tissue or are cells manipulated to exhibit a cancer phenotype.

32. (canceled)

33. A method for profiling Huntington's Disease, comprising:
a) obtaining one or more cells expressing mutant Huntington protein,
b) labeling the one or more cells expressing mutant Huntington protein with a panel of fluorescently labeled reagents, thereby producing one or more fluorescently labeled cells, wherein each fluorescently labeled reagent is specific for a biomarker, and wherein the panel of fluorescently labeled reagents detects five or more biomarkers, and wherein the detection of a biomarker is a read-out of one or more features of a cellular systems biology profile, and wherein the one or more features can be the same or different for each biomarker detected;
c) imaging the one or more fluorescently labeled cells with at least one optical mode, wherein the imaging produces a first set of data; and
d) analyzing the first set of data to read-out the five or more biomarkers, wherein the combination of the five or more biomarkers generates a cellular systems biology profile of the one or more cells expressing mutant huntington protein,
thereby profiling the Huntington's Disease.

34. A method for analyzing one or more cells for the presence of a disease state comprising:
a) obtaining one or more cells to test for the presence of a disease state;
b) labeling the one or more cells with a panel of fluorescently labeled reagents, thereby producing one or more fluorescently labeled cells, wherein each fluorescently labeled reagent is specific for a biomarker, the panel of fluorescently labeled reagents detects at least five different biomarkers, and the detection of a biomarker provides a read-out of one or more features of the one or more cells;
c) imaging the one or more fluorescently labeled cells with at least one optical mode, wherein the imaging produces a first set of data;
d) analyzing the first set of data to read-out the features of the five or more biomarkers, wherein the combination of the features of the five or more biomarkers generates a cellular systems biology profile of the one or more cells; and
e) comparing the cellular systems biology profile of the one or more cells with a control cellular systems biology profile, thereby analyzing one or more cells for the presence of a disease state.

35. (canceled)

36. (canceled)
